(19) United States
(12) Patent Application Publication
(10) Pub. No.: US 2025/0387417 A1
(43) Pub. Date: Dec. 25, 2025

(54) CANNABINOID DERIVATIVES AND COMPOSITIONS COMPRISING SAME

(71) Applicant: CANNASOUL ANALYTICS LTD., Caesarea (IL)

(72) Inventors: Shmuel MANDEL, Atlit (IL); Rotem PERRY-FEIGENBAUM, Bazra (IL); David MEIRI, Tzur Moshe (IL); Boaz MUSAFIA, Shavei Tzion (IL); Konstantin ULANENKO, Netanya (IL); Irit VENTURA, Zikhron Yaacov (IL)

(21) Appl. No.: 18/879,213

(22) PCT Filed: Jun. 28, 2023
(86) PCT No.: PCT/IL2023/050664

§ 371 Date: Dec. 26, 2024

Related U.S. Application Data
(60) Provisional application No. 63/356,521, filed on Jun. 29, 2022.

(51) Int. Cl. A61K 31/00; A61P 1/00; C07B 59/00; C07C 69/76; C07D 311/64; C07D 311/80

(52) U.S. Cl. CPC A61K 31/658; A61P 1/00; C07B 59/001; C07C 69/76; C07D 311/64; C07D 311/80; C07B 2200/05; C07C 2601/16

(57) ABSTRACT

The present invention provides cannabinoid derivatives, pharmaceutical compositions comprising same, and methods of use thereof as medicaments.

### FIELD OF THE INVENTION

The present invention relates to new cannabinoid derivatives, methods for their preparation, pharmaceutical compositions comprising same and uses thereof.

### BACKGROUND OF THE INVENTION

The cannabis plant has been used as an herbal remedy for centuries. It contains more than 500 different active components including phytocannabinoids, the most prevalent ones are delta-9-tetrahydrocannabinol (Δ9-THC) and cannabidiol (CBD), terpenes and flavonoids. The medical use of the cannabis plant is still controversial. However, to date, there is a variety of conditions including pain for which certain phytocannabinoids have been proven effective.

WO 2022/144878 describes a pharmaceutical cannabinoid composition comprising a compound having a structure represented by Formula II:

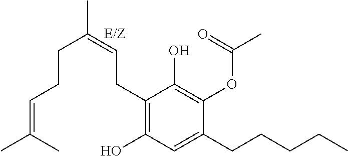 [SMILES: CCCCCc1cc(O)c(C/C=C(/C)CCC=C(C)C)c(O)c1OC(C)=O]

or a pharmaceutically acceptable salt thereof, and methods of use of said compound or salt or a pharmaceutical composition comprising same for the treatment of a disease related to estrogen receptor (ER) as well as for increasing the sensitivity of a subject afflicted with an ER-related disease to an ER activation inhibitor.

Inflammatory bowel disease (IBD) is a generic classification which includes several forms of inflammatory diseases and conditions affecting various parts of the gastrointestinal (GI) tract, such as the colon and small intestine. The main forms of IBD include Crohn's disease and ulcerative colitis, among other forms of colitis. IBD's manifestations typically include diarrhea, nausea, vomiting, abdominal cramps, and uncontrolled pain. The most commonly used conventional therapies are anti-inflammatory medicaments such as corticosteroids, and immunosuppressives. While these therapies have shown efficacy, in most cases the efficacy is only partial with significant side effects being experienced by patients.

U.S. 2017/0112787 describes a method of improving immune homeostasis in the gut of a subject suffering from an autoimmune disease characterized by inflammation of the gut comprising administering to the subject an effective amount of a cannabinoid receptor ligand, e.g., a cannabinoid, to improve immune homeostasis in the gut of the subject, wherein the gut includes the gastrointestinal tract as well as organs served by the blood supply to the gut.

There remains an unmet need for cannabinoid compounds having therapeutic benefits and reduced adverse effects.

### SUMMARY OF THE INVENTION

The present invention is directed to novel cannabinoid derivatives. The present invention is further directed to methods of preparing said novel cannabinoid derivatives, pharmaceutical compositions comprising same, and use thereof in the treatment of various diseases and disorders.

According to one aspect, the present invention provides a compound represented by the structure of formula I:

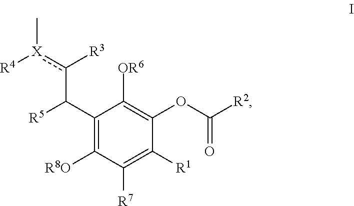

wherein
X is C, CH, or CD;
R¹ is C₁-C₁₂ alkyl, C₂-C₁₂ alkenyl, or C₂-C₁₂ alkynyl;
R² is C₁-C₁₀ alkyl, C₂-C₁₀ alkenyl, or C₂-C₁₀ alkynyl;
R³ and R⁷ are each independently H, deuterium, hydroxyl, halogen, nitro, or cyano;
R⁴ is H, deuterium, C₁-C₁₂ alkyl, C₂-C₁₂ alkenyl, or C₂-C₁₂ alkynyl;
R⁵ is H, deuterium, or R⁴ and R⁵ together form an aromatic or non-aromatic ring which is optionally substituted by C₁-C₄ alkyl, C₂-C₄ alkenyl, or hydroxyl;
R⁶ is H, deuterium, or R⁶ is absent and the oxygen attached thereto together with X form a six-membered heterocycle;
R⁸ is H, deuterium, or R⁸ together with R⁵ form a six-membered heterocycle which is optionally substituted by C₁-C₄ alkyl or C₂-C₄ alkenyl; and
the dotted line represents an optional second carbon-carbon bond, wherein each of the alkyl, alkenyl, or alkynyl is optionally deuterated, with the proviso that when X is C; R² is methyl; R³, R⁵, R⁶, R⁷, and R⁸ are each H; R⁴ is —CH₂CH₂CH═C(CH₃)₂; and the dotted line represents a second carbon-carbon bond, then R¹ is not n-pentyl,
including salts, hydrates, solvates, polymorphs, optical isomers, geometrical isomers, enantiomers, and diastereomers. Each possibility represents a separate embodiment.

In some embodiments, the present invention provides a compound represented by the structure of formula I, wherein X is C. In other embodiments, the present invention provides a compound represented by the structure of formula I, wherein X is CH.

In several embodiments, the present invention provides a compound represented by the structure of formula I, wherein R¹ is C₁-C₁₂ alkyl. In other embodiments, R¹ is C₁-C₁₀ alkyl. In particular embodiments, R¹ is selected from the group consisting of methyl, ethyl, propyl, butyl, pentyl, hexyl, heptyl, octyl, 1,1-dimethyl pentyl, 1-methyl pentyl, 1-methyl heptyl, 1,1-dimethyl heptyl, and 1-phenyl ethyl. Each possibility represents a separate embodiment.

In certain embodiments, the present invention provides a compound represented by the structure of formula I, wherein R² is selected from the group consisting of methyl, ethyl, and isopropyl. Each possibility represents a separate embodiment. In other embodiments, the present invention provides a compound represented by the structure of formula I, wherein R² is trifluoromethyl. In yet other embodiments, the present invention provides a compound represented by the structure of formula I, wherein R² is CD₃. In further embodiments, the present invention provides a compound represented by the structure of formula I, wherein R² is 1-propylbutyl.

In various embodiments, the present invention provides a compound represented by the structure of formula I, wherein R³ is hydrogen. In other embodiments, the present invention provides a compound represented by the structure of formula I, wherein R³ is D.

In some embodiments, the present invention provides a compound represented by the structure of formula I, wherein R⁴ is C₁-C₄ alkyl. In further embodiments, the present invention provides a compound represented by the structure of formula I, wherein R⁴ is methyl.

In certain embodiments, R⁴ and R⁵ together form a terpineol or a limonene.

In various embodiments, the present invention provides a compound represented by the structure of formula I, wherein R⁶ is H. In further embodiments, the present invention provides a compound represented by the structure of formula I, wherein R⁶ is absent and the oxygen attached thereto together with X form a dihydropyran.

In some embodiments, the present invention provides a compound represented by the structure of formula I, wherein R⁷ is D. In other embodiments, the present invention provides a compound represented by the structure of formula I, wherein R⁷ is halogen, preferably F.

In certain embodiments, the present invention provides a compound represented by the structure of formula I, wherein R⁸ is H. In other embodiments, R⁸ together with R⁵ form a dihydropyran or a 2H-pyran.

In additional embodiments, the dotted line is absent. In other embodiments, the dotted line represents a second carbon-carbon bond.

In some embodiments, the compound represented by the structure of formula I is a compound represented by the structure of formula Ia:

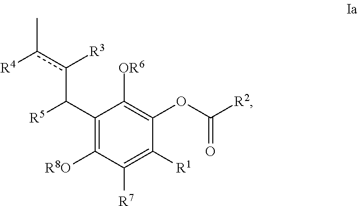

wherein
R¹ is C₁-C₁₂ alkyl, C₂-C₁₂ alkenyl, or C₂-C₁₂ alkynyl;
R² is C₁-C₁₀ alkyl, C₂-C₁₀ alkenyl, or C₂-C₁₀ alkynyl;
R³ and R⁷ are each independently H, deuterium, hydroxyl, halogen, nitro, or cyano;
R⁴ is H, deuterium, C₁-C₁₂ alkyl, C₂-C₁₂ alkenyl, or C₂-C₁₂ alkynyl;
R⁵, R⁶, and R⁸ are each independently H or deuterium; and
the dotted line represents an optional second carbon-carbon bond, wherein each of the alkyl, alkenyl, or alkynyl is optionally deuterated, with the proviso that when R² is methyl, R³, R⁵, R⁶, R⁷, and R⁸ are each H, R⁴ is —CH₂CH₂CH═C(CH₃)₂, and the dotted line represents a second carbon-carbon bond, then R¹ is not n-pentyl,
including salts, hydrates, solvates, polymorphs, optical isomers, geometrical isomers, enantiomers, and diastereomers. Each possibility represents a separate embodiment.

In further embodiments, the compound represented by the structure of formula I is a compound represented by the structure of formula Ib:

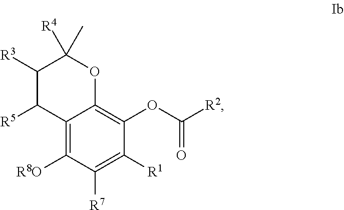

wherein
   R¹ is C₁-C₁₂ alkyl, C₂-C₁₂ alkenyl, or C₂-C₁₂ alkynyl;
   R² is C₁-C₁₀ alkyl, C₂-C₁₀ alkenyl, or C₂-C₁₀ alkynyl;
   R³ and R⁷ are each independently H, deuterium, hydroxyl, halogen, nitro, or cyano;
   R⁴ is H, deuterium, C₁-C₁₂ alkyl, C₂-C₁₂ alkenyl, or C₂-C₁₂ alkynyl; and
   R⁵ and R⁸ are each independently H or deuterium;
   wherein each of the alkyl, alkenyl, or alkynyl is optionally deuterated,
including salts, hydrates, solvates, polymorphs, optical isomers, geometrical isomers, enantiomers, and diastereomers. Each possibility represents a separate embodiment.

In additional embodiments, the compound represented by the structure of formula I is a compound represented by the structure of formula Ic:

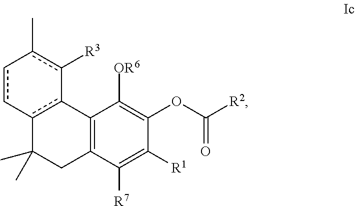

wherein
   R¹ is C₁-C₁₂ alkyl, C₂-C₁₂ alkenyl, or C₂-C₁₂ alkynyl;
   R² is C₁-C₁₀ alkyl, C₂-C₁₀ alkenyl, or C₂-C₁₀ alkynyl;
   R³ and R⁷ are each independently H, deuterium, hydroxyl, halogen, nitro, or cyano;
   R⁶ is H or deuterium; and
   the dotted lines represent optional second carbon-carbon bonds, wherein each of the alkyl, alkenyl, or alkynyl is optionally deuterated,
including salts, hydrates, solvates, polymorphs, optical isomers, geometrical isomers, enantiomers, and diastereomers. Each possibility represents a separate embodiment.

In various embodiments, the compound represented by the structure of formula I is a compound represented by the structure of formula Id:

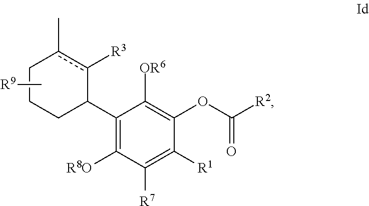

wherein
   R¹ is C₁-C₁₂ alkyl, C₂-C₁₂ alkenyl, or C₂-C₁₂ alkynyl;
   R² is C₁-C₁₀ alkyl, C₂-C₁₀ alkenyl, or C₂-C₁₀ alkynyl;
   R³ and R⁷ are each independently H, deuterium, hydroxyl, halogen, nitro, or cyano;
   R⁶ and R⁸ are each independently H or deuterium;
   R⁹ is C₁-C₄ alkyl or C₂-C₄ alkenyl; and
   the dotted line represents an optional second carbon-carbon bond, wherein each of the alkyl, alkenyl, or alkynyl is optionally deuterated,
including salts, hydrates, solvates, polymorphs, optical isomers, geometrical isomers, enantiomers, and diastereomers. Each possibility represents a separate embodiment.

In one embodiment, the present invention provides a compound represented by the structure of formula I, wherein X is C; R¹ and R² are each methyl; R³, R⁵, R⁶, R⁷, and R⁸ are each H; R⁴ is —CH₂CH₂CH═C(CH₃)₂; and the dotted line represents a second carbon-carbon bond. In another embodiment, the present invention provides a compound represented by the structure of formula I, wherein X is C; R¹ is ethyl; R² is methyl; R³, R⁵, R⁶, R⁷, and R⁸ are each H; R⁴ is —CH₂CH₂CH═C(CH₃)₂; and the dotted line represents a second carbon-carbon bond. In yet another embodiment, the present invention provides a compound represented by the structure of formula I, wherein X is C; R¹ is propyl; R² is methyl; R³, R⁵, R⁶, R⁷, and R⁸ are each H; R⁴ is —CH₂CH₂CH═C(CH₃)₂, and the dotted line represents a second carbon-carbon bond. In other embodiments, the present invention provides a compound represented by the structure of formula I, wherein X is C; R¹ is butyl; R² is methyl; R³, R⁵, R⁶, R⁷, and R⁸ are each H; R⁴ is —CH₂CH₂CH═C(CH₃)₂; and the dotted line represents a second carbon-carbon bond. In further embodiments, the present invention provides a compound represented by the structure of formula I, wherein X is C; R¹ is hexyl; R² is methyl; R³, R⁵, R⁶, R⁷, and R⁸ are each H; R⁴ is —CH₂CH₂CH═C(CH₃)₂; and the dotted line represents a second carbon-carbon bond. In additional embodiments, the present invention provides a compound represented by the structure of formula I, wherein X is C; R¹ is heptyl; R² is methyl; R³, R⁵, R⁶, R⁷, and R⁸ are each H; R⁴ is —CH₂CH₂CH═C(CH₃)₂, and the dotted line represents a second carbon-carbon bond. In other embodiments, the present invention provides a compound represented by the structure of formula I, wherein X is C; R¹ is 1,1-dimethyl heptyl; R² is methyl; R³, R⁵, R⁶, R⁷, and R⁸ are each H; R⁴ is —CH₂CH₂CH═C(CH₃)₂; and the dotted line represents a second carbon-carbon bond.

In some embodiments, the present invention provides a compound represented by the structure of formula I, wherein X is C; R¹ is pentyl; R² is trifluoromethyl; R³, R⁵, R⁶, R⁷, and R⁸ are each H; R⁴ is —CH₂CH₂CH═C(CH₃)₂; and the dotted line represents a second carbon-carbon bond. In other embodiments, the present invention provides a compound represented by the structure of formula I, wherein X is C; R¹ is pentyl; R² is ethyl; R³, R⁵, R⁶, R⁷, and R⁸ are each H; R⁴ is —CH₂CH₂CH═C(CH₃)₂; and the dotted line represents a second carbon-carbon bond. In yet other embodiments, the present invention provides a compound represented by the structure of formula I, wherein X is C; R¹ is pentyl; R² is isopropyl; R³, R⁵, R⁶, R⁷, and R⁸ are each H; R⁴ is —CH₂CH₂CH═C(CH₃)₂; and the dotted line represents a second carbon-carbon bond. In further embodiments, the present invention provides a compound represented by the structure of formula I, wherein X is C; R¹ is pentyl; R² is CD₃; R³, R⁵, R⁶, R⁷, and R⁸ are each H; R⁴ is —CH₂CH₂CH═C(CH₃)₂; and the dotted line represents a second carbon-carbon bond. In additional embodiments, the present invention provides a compound represented by the structure of formula I, wherein X is C; R¹ is pentyl; R² is 1-propylbutyl; R³, R⁵, R⁶, R⁷, and R⁸ are each H; R⁴ is —CH₂CH₂CH═C(CH₃)₂; and the dotted line represents a second carbon-carbon bond.

In one embodiment, the present invention provides a compound represented by the structure of formula I, wherein X is C; R¹ is pentyl; R² is methyl; R³ is deuterium; R⁵, R⁶, R⁷, and R⁸ are each H; R⁴ is —CH₂CH₂CH═C(CH₃)₂, and the dotted line represents a second carbon-carbon bond.

In another embodiment, the present invention provides a compound represented by the structure of formula I, wherein X is C; R¹ is pentyl; R² is trifluoromethyl; R³, R⁵, R⁶, R⁷, and R⁸ are each H; R⁴ is methyl; and the dotted line represents a second carbon-carbon bond. In additional embodiments, the present invention provides a compound represented by the structure of formula I, wherein X is C; R¹ is hexyl; R² is trifluoromethyl; R³, R⁵, R⁶, R⁷, and R⁸ are each H; R⁴ is methyl; and the dotted line represents a second carbon-carbon bond. In various embodiments, the present invention provides a compound represented by the structure of formula I, wherein X is C; R¹ is heptyl; R² is trifluoromethyl; R³, R⁵, R⁶, R⁷, and R⁸ are each H; R⁴ is methyl; and the dotted line represents a second carbon-carbon bond. In further embodiments, the present invention provides a compound represented by the structure of formula I, wherein X is C; R¹ is propyl; R² is trifluoromethyl; R³, R⁵, R⁶, R⁷, and R⁸ are each H; R⁴ is methyl; and the dotted line represents a second carbon-carbon bond. In particular embodiments, the present invention provides a compound represented by the structure of formula I, wherein X is C; R¹ is 1,1-dimethyl heptyl; R² is trifluoromethyl; R³, R⁵, R⁶, R⁷, and R⁸ are each H; R⁴ is methyl; and the dotted line represents a second carbon-carbon bond.

In certain embodiments, the present invention provides a compound represented by the structure of formula I, wherein X is C; R¹ is 1,1-dimethyl heptyl; R² and R⁴ are each methyl; R³, R⁵, R⁶, R⁷, and R⁸ are each H; and the dotted line represents a second carbon-carbon bond. In other embodiments, the present invention provides a compound represented by the structure of formula I, wherein X is C; R¹ is hexyl; R² and R⁴ are each methyl; R³, R⁵, R⁶, R⁷, and R⁸ are each H; and the dotted line represents a second carbon-carbon bond. In further embodiments, the present invention provides a compound represented by the structure of formula I, wherein X is C; R¹ is heptyl; R² and R⁴ are each methyl; R³, R⁵, R⁶, R⁷, and R⁸ are each H; and the dotted line represents a second carbon-carbon bond. In some embodiments, the present invention provides a compound represented by the structure of formula I, wherein X is C; R¹ is propyl; R² and R⁴ are each methyl; R³, R⁵, R⁶, R⁷, and R⁸ are each H; and the dotted line represents a second carbon-carbon bond. In other embodiments, the present invention provides a compound represented by the structure of formula I, wherein X is C; R¹ is 1,1-dimethyl heptyl; R² is trifluoromethyl; R³, R⁵, R⁶, R⁷, and R⁸ are each H; R⁴ is —CH₂CH₂CH═C(CH₃)₂; and the dotted line represents a second carbon-carbon bond.

In one embodiment, the present invention provides a compound represented by the structure of formula I, wherein X is C; R¹ is pentyl; R² is methyl; R³, R⁵, R⁶, and R⁸ are each H; R⁴ is —CH₂CH₂CH═C(CH₃)₂; R⁷ is deuterium; and the dotted line represents a second carbon-carbon bond. In another embodiment, the present invention provides a compound represented by the structure of formula I, wherein X is C; R¹ is pentyl; R² is methyl; R³, R⁵, R⁶, and R⁸ are each H; R⁴ is —CH₂CH₂CH═C(CH₃)₂; R⁷ is F; and the dotted line represents a second carbon-carbon bond.

In some embodiments, the present invention provides a compound represented by the structure of formula I, wherein X is C; R¹ is pentyl; R² is methyl; R³, R⁵, R⁶, and R⁸ are each H; R⁴ is —CH₂CH₂CH═C(CH₃)₂; R⁷ is deuterium; and the dotted line represents a second carbon-carbon bond.

In other embodiments, the present invention provides a compound represented by the structure of formula I, wherein X is C; R¹ is pentyl; R² is methyl; R³, R⁶, R⁷, and R⁸ are each H; R⁴ and R⁵ together form a non-aromatic six-membered ring which is substituted by isopropenyl; and the dotted line represents a second carbon-carbon bond.

In further embodiments, the present invention provides a compound represented by the structure of formula I, wherein X is C; R¹ is pentyl; R² is methyl; R³, R⁶, R⁷, and R⁸ are each H; R⁴ and R⁵ together form a non-aromatic six-membered ring which is substituted by —C(CH₃)₂OH; and the dotted line represents a second carbon-carbon bond.

In various embodiments, the present invention provides a compound represented by the structure of formula I, wherein X is C; R¹ is pentyl; R² is CF3; R³, R⁶, R⁷, and R⁸ are each H; R⁴ and R⁵ together form a non-aromatic six-membered ring which is substituted by —C(CH₃)₂OH; and the dotted line represents a second carbon-carbon bond.

In some embodiments, the present invention provides a compound represented by the structure of formula I, wherein X is C; R¹ is propyl; R² is methyl; R³, R⁶, R⁷, and R⁸ are each H; R⁴ and R⁵ together form a non-aromatic six-membered ring which is substituted by —C(CH₃)₂OH; and the dotted line represents a second carbon-carbon bond.

In other embodiments, the present invention provides a compound represented by the structure of formula I, wherein X is C; R¹ is pentyl; R² is methyl; R³, R⁶, and R⁷ are each H; R⁸ together with R⁵ form a dihydropyran substituted by 2,2 dimethyl; and R⁴ and R⁵ together form a cyclohexene. It is to be understood that when R⁴ and R⁵ together form a cyclohexene then the dotted line represents a second carbon-carbon bond.

In yet other embodiments, the present invention provides a compound represented by the structure of formula I, wherein X is C; R¹ is pentyl; R² is methyl; R³, R⁶, and R⁷ are each H; R⁸ together with R⁵ form a 2H-pyran substituted by 2,2 dimethyl; and R⁴ and R⁵ together form a benzene. It is to be understood that when R⁴ and R⁵ together form a benzene then the dotted line represents a second carbon-carbon bond.

In further embodiments, the present invention provides a compound represented by the structure of formula I, wherein R¹ is pentyl; R² is methyl; R³, R⁷, and R⁸ are each H; R⁴ is —CH₂CH₂CH═C(CH₃)₂; R⁶ is absent and the oxygen attached thereto together with X form a dihydropyran; and the dotted line is absent.

It is to be understood for all compounds of the present invention encompassed by the structure of formula I that when X is C; R² is methyl; R³, R⁵, R⁶, R⁷, and R⁸ are each H; R⁴ is —CH₂CH₂CH═C(CH₃)₂; and the dotted line represents a second carbon-carbon bond, then R¹ is not n-pentyl. Likewise, for the compounds of the present invention encompassed by the structure of formula Ia, when R² is methyl; R³, R⁵, R⁶, R⁷, and R⁸ are each H; R⁴ is —CH₂CH₂CH═C(CH₃)₂; and the dotted line represents a second carbon-carbon bond, then R¹ is not n-pentyl.

Representative and non-limiting examples of such structures are compounds selected from the group consisting of compounds 1-33:

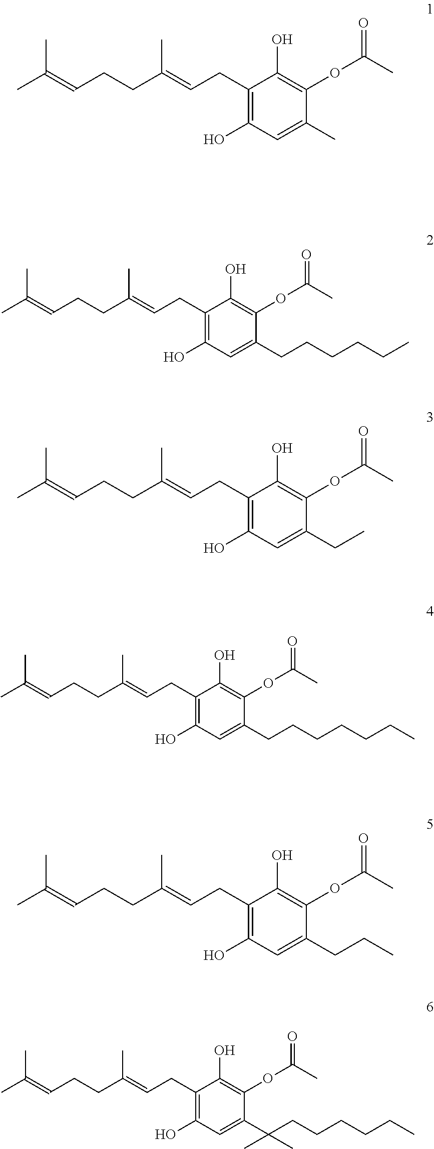

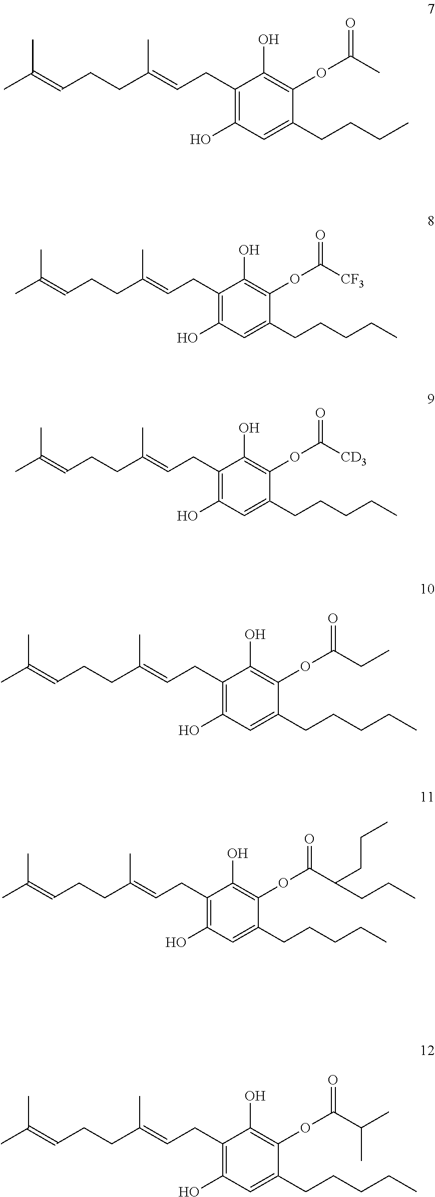

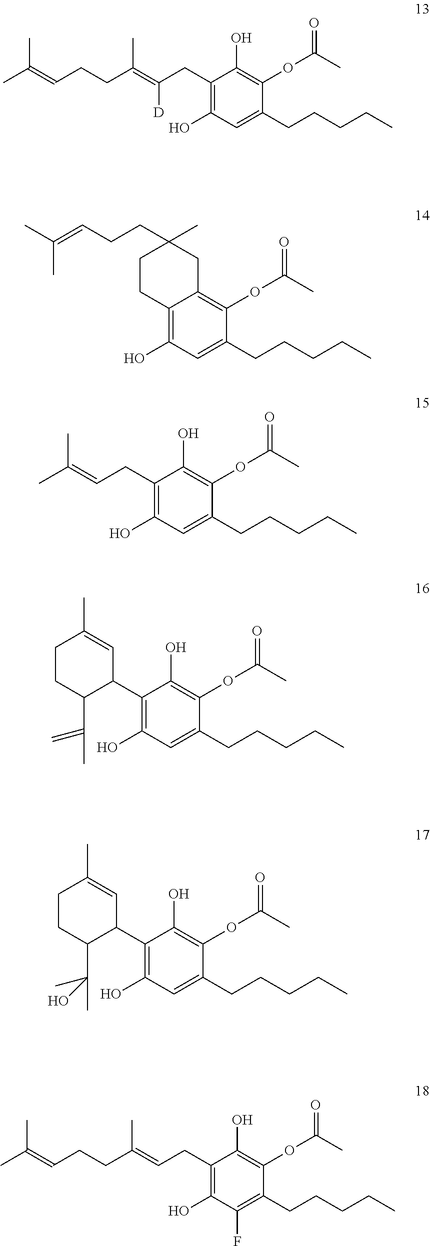

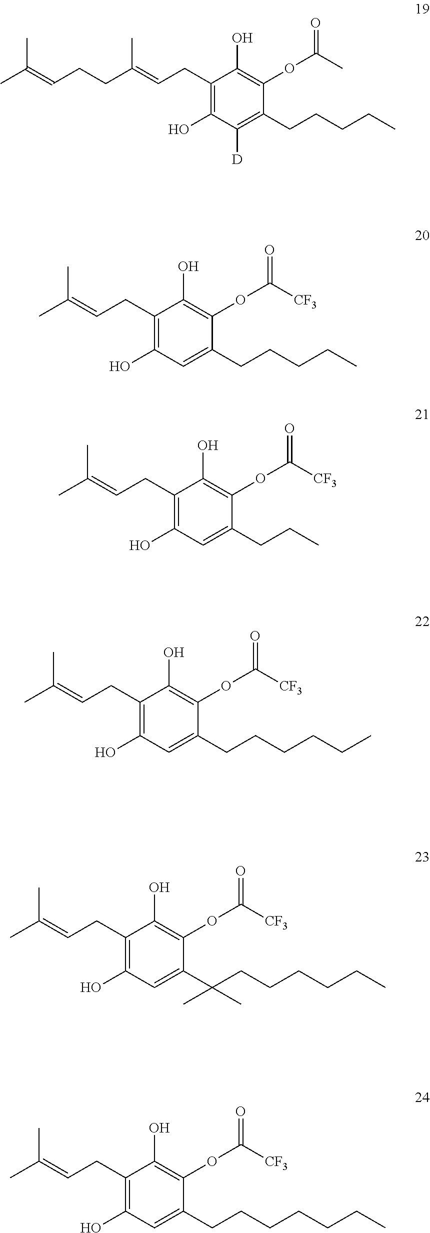

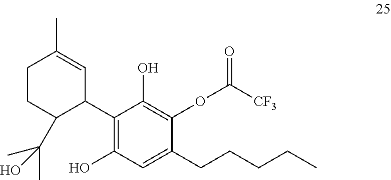 [SMILES: CCCCCc1cc(O)c(C2C=C(C)CCC2C(C)(C)O)c(O)c1OC(=O)C(F)(F)F]

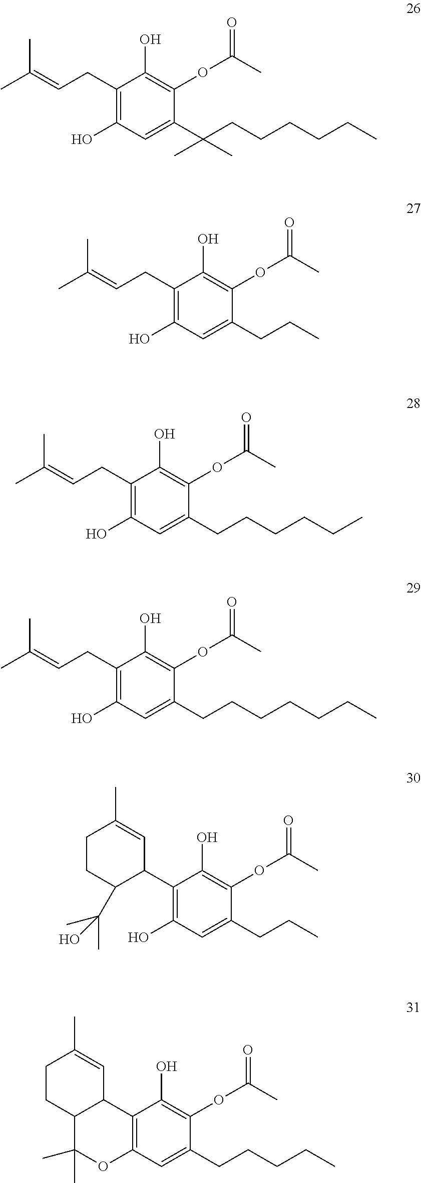

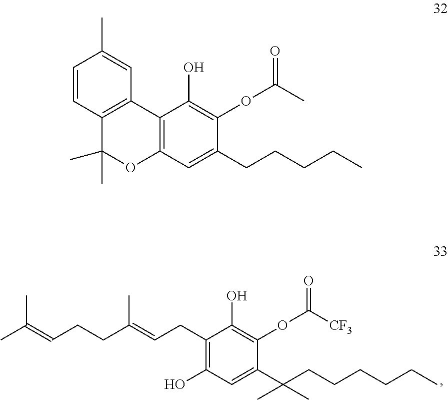

including salts, hydrates, solvates, polymorphs, optical isomers, geometrical isomers, enantiomers, and diastereomers. Each possibility represents a separate embodiment.

The present invention further provides a pharmaceutical composition comprising a therapeutically effective amount of at least one compound represented by the structure of formula I and a pharmaceutically acceptable carrier or excipient. In one embodiment, the pharmaceutical composition comprises the compound disclosed herein as the sole active ingredient. In another embodiment, the pharmaceutical composition comprises the compound represented by the structure of formula I in combination with one or more cannabinoids, terpenes, terpenoids, flavonoids, oils, nitrogenous compounds, amino acids, proteins, glycoproteins, sugars, hydrocarbons, fatty acids, esters, lactones, steroids, non-cannabinoid phenols, and a mixture or combination thereof. Each possibility represents a separate embodiment. In other embodiments, the pharmaceutical composition comprising a compound represented by the structure of formula I is adapted for administration in combination with an ER activation inhibitor. In a particular embodiment, the ER activation inhibitor is tamoxifen.

In some embodiments, the pharmaceutically acceptable carrier or excipient comprises at least one of a binder, a filler, a diluent, a surfactant or emulsifier, a glidant or lubricant, a buffering or pH adjusting agent, a tonicity enhancing agent, a wetting agent, a chelating agent, a preservative, an antioxidant, a flavoring agent, a colorant, and a mixture or combination thereof. Each possibility represents a separate embodiment. In one particular embodiment, the pharmaceutically acceptable carrier is a lipid carrier.

In other embodiments, the pharmaceutical composition is in a form selected from the group consisting of tablet, pill, capsule (e.g., soft or hard gelatin capsule), pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, ointment, suppository, a gel, and a cream. Each possibility represents a separate embodiment.

In further embodiments, the pharmaceutical composition is formulated (or adapted) for administration via a route selected from the group consisting of oral, topical, transdermal, intra-arterial, sub-lingual, intranasal, intraperitoneal, intramuscular, subcutaneous, intravenous, and intra-alveolar. Each possibility represents a separate embodiment.

In some embodiments, the present invention provides a pharmaceutical composition comprising a therapeutically effective amount of at least one compound represented by the structure of formula I and a pharmaceutically acceptable carrier or excipient for use as a medicament.

In particular embodiments, the present invention provides a pharmaceutical composition comprising a therapeutically effective amount of at least one compound represented by the structure of formula I and a pharmaceutically acceptable carrier or excipient for use in treating an estrogen receptor (ER)-related disease or disorder.

In further embodiments, the present invention provides a method of treating an estrogen receptor (ER)-related disease or disorder, the method comprising administering to a subject in need thereof a pharmaceutical composition comprising a therapeutically effective amount of at least one compound represented by the structure of formula I and a pharmaceutically acceptable carrier or excipient.

In specific embodiments, the estrogen receptor (ER)-related disease or disorder is selected from the group consisting of breast cancer, ovarian cancer, uterine serous carcinoma, colon cancer, prostate cancer, polycystic ovary syndrome, endometrial cancer, endometriosis, fibrosis, dysmenorrhea, precocious puberty, and gynecomastia. Each possibility represents a separate embodiment.

In further embodiments, the present invention provides a pharmaceutical composition comprising a therapeutically effective amount of at least one compound represented by the structure of formula I and a pharmaceutically acceptable carrier or excipient for use in treating inflammatory bowel disease.

In further embodiments, the present invention provides a method of treating inflammatory bowel disease, the method comprising administering to a subject in need thereof a pharmaceutical composition comprising a therapeutically effective amount of at least one compound represented by the structure of formula I and a pharmaceutically acceptable carrier or excipient.

In specific embodiments, the inflammatory bowel disease is selected from the group consisting of Crohn's disease, ulcerative colitis, granulomatous colitis, lymphocyte colitis, collagenous colitis, diversion colitis, and coeliac disease. Each possibility represents a separate embodiment.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1. shows the effects of (E)-3-(3,7-dimethylocta-2,6-dien-1-yl)-6-hexyl-2,4-dihydroxyphenyl acetate (CF2), a compound of formula 17 (17), and a compound of formula 15 (15) on MCP-1 secretion in an intestinal inflammation model using LPS. NT represents no treatment and Rosi represents Rosiglitazone (positive control).

### DETAILED DESCRIPTION OF THE INVENTION

The present invention relates to novel cannabinoids and method of their preparation as well as pharmaceutical compositions comprising same.

The novel cannabinoids of the present invention are designed to treat various diseases and disorders including, but not limited to, estrogen receptor (ER)-related disease or disorder and inflammatory bowel disease.

### Cannabinoids

The present invention provides compounds that are represented by the structure of formula I:

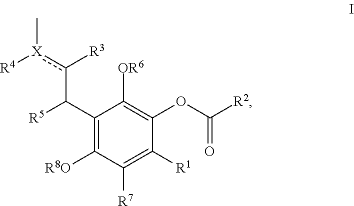

wherein
X is C, CH, or CD;
R¹ is C₁-C₁₂ alkyl, C₂-C₁₂ alkenyl, or C₂-C₁₂ alkynyl;
R² is C₁-C₁₀ alkyl, C₂-C₁₀ alkenyl, or C₂-C₁₀ alkynyl;
R³ and R⁷ are each independently H, deuterium, hydroxyl, halogen, nitro, or cyano;
R⁴ is H, deuterium, C₁-C₁₂ alkyl, C₂-C₁₂ alkenyl, or C₂-C₁₂ alkynyl;
R⁵ is H, deuterium, or R⁴ and R⁵ together form an aromatic or non-aromatic ring which is optionally substituted by C₁-C₄ alkyl, C₂-C₄ alkenyl, or hydroxyl;
R⁶ is H, deuterium, or R⁶ is absent and the oxygen attached thereto together with X form a six-membered heterocycle;
R⁸ is H, deuterium, or R⁸ together with R⁵ form a six-membered heterocycle which is optionally substituted by C₁-C₄ alkyl or C₂-C₄ alkenyl; and
the dotted line represents an optional second carbon-carbon bond, wherein each of the alkyl, alkenyl, or alkynyl is optionally deuterated, with the proviso that when X is C; R² is methyl; R³, R⁵, R⁶, R⁷, and R⁸ are each H; R⁴ is —CH₂CH₂CH═C(CH₃)₂; and the dotted line represents a second carbon-carbon bond, then R¹ is not n-pentyl,
including salts, hydrates, solvates, polymorphs, optical isomers, geometrical isomers, enantiomers, and diastereomers. Each possibility represents a separate embodiment.

The present invention further provides currently preferred embodiments wherein formula I comprises the following substitutions with the proviso that when X is C; R² is methyl; R³, R⁵, R⁶, R⁷, and R⁸ are each H; R⁴ is —CH₂CH₂CH═C(CH₃)₂; and the dotted line represents a second carbon-carbon bond, then R¹ is not n-pentyl:
1. According to some embodiments, X is C or CH.
2. According to some embodiments, R¹ is C₁-C₁₂ alkyl, preferably C₁-C₁₀ alkyl.
3. According to some embodiments, R¹ is selected from the group consisting of propyl, butyl, pentyl, hexyl, heptyl, octyl, 1,1-dimethyl pentyl, 1-methyl pentyl, 1-methyl heptyl, 1,1-dimethyl heptyl, and 1-phenyl ethyl. Each possibility represents a separate embodiment.
4. According to some embodiments, R² is methyl, ethyl, or isopropyl. Each possibility represents a separate embodiment.
5. According to some embodiments, R² is trifluoromethyl.
6. According to some embodiments, R² is CD₃.
7. According to some embodiments, R² is 1-propylbutyl (4-heptyl).
8. According to some embodiments, R³ is H or D.
9. According to some embodiments, R⁴ is C₁-C₄ alkyl, preferably methyl.
10. According to some embodiments, R⁴ and R⁵ together form a terpineol or a limonene.
11. According to some embodiments, R⁷ is hydrogen.
12. According to some embodiments, R⁶ is absent and the oxygen attached thereto together with X form a dihydropyran.
13. According to some embodiments, R⁷ is D.
14. According to some embodiments, R⁷ is halogen, preferably F.
15. According to some embodiments, R⁸ is hydrogen.
16. According to some embodiments, R⁸ together with R⁵ form a dihydropyran or a 2H-pyran.

According to certain aspects and embodiments, the dotted line is absent. According to other embodiments, the dotted line represents a double carbon-carbon bond.

In some embodiments, the compound represented by the structure of formula I is a compound of formula Ia:

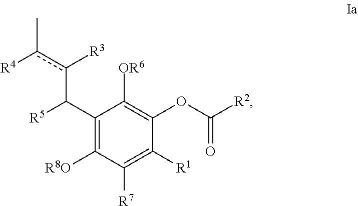

wherein
   R¹ is C₁-C₁₂ alkyl, C₂-C₁₂ alkenyl, or C₂-C₁₂ alkynyl;
   R² is C₁-C₁₀ alkyl, C₂-C₁₀ alkenyl, or C₂-C₁₀ alkynyl;
   R³ and R⁷ are each independently H, deuterium, hydroxyl, halogen, nitro, or cyano;
   R⁴ is H, deuterium, C₁-C₁₂ alkyl, C₂-C₁₂ alkenyl, or C₂-C₁₂ alkynyl;
   R⁵, R⁶, and R⁸ are each independently H or deuterium; and
   the dotted line represents an optional second carbon-carbon bond, wherein each of the alkyl, alkenyl, or alkynyl is optionally deuterated, with the proviso that when R² is methyl, R³, R⁵, R⁶, R⁷, and R⁸ are each H, R⁴ is —CH₂CH₂CH═C(CH₃)₂, and the dotted line represents a second carbon-carbon bond, then R¹ is not n-pentyl,
including salts, hydrates, solvates, polymorphs, optical isomers, geometrical isomers, enantiomers, and diastereomers. Each possibility represents a separate embodiment.

In further embodiments, the compound represented by the structure of formula I is a compound of formula Ib:

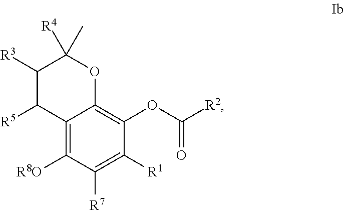

wherein
   R¹ is C₁-C₁₂ alkyl, C₂-C₁₂ alkenyl, or C₂-C₁₂ alkynyl;
   R² is C₁-C₁₀ alkyl, C₂-C₁₀ alkenyl, or C₂-C₁₀ alkynyl;
   R³ and R⁷ are each independently H, deuterium, hydroxyl, halogen, nitro, or cyano;
   R⁴ is H, deuterium, C₁-C₁₂ alkyl, C₂-C₁₂ alkenyl, or C₂-C₁₂ alkynyl; and
   R⁵ and R⁸ are each independently H or deuterium;
   wherein each of the alkyl, alkenyl, or alkynyl is optionally deuterated,
including salts, hydrates, solvates, polymorphs, optical isomers, geometrical isomers, enantiomers, and diastereomers. Each possibility represents a separate embodiment.

In additional embodiments, the compound represented by the structure of formula I is a compound of formula Ic:

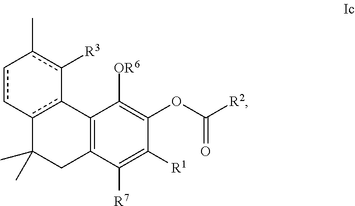

wherein
   R¹ is C₁-C₁₂ alkyl, C₂-C₁₂ alkenyl, or C₂-C₁₂ alkynyl;
   R² is C₁-C₁₀ alkyl, C₂-C₁₀ alkenyl, or C₂-C₁₀ alkynyl;
   R³ and R⁷ are each independently H, deuterium, hydroxyl, halogen, nitro, or cyano;
   R⁶ is H or deuterium; and
   the dotted lines represent optional second carbon-carbon bonds, wherein each of the alkyl, alkenyl, or alkynyl is optionally deuterated,
including salts, hydrates, solvates, polymorphs, optical isomers, geometrical isomers, enantiomers, and diastereomers. Each possibility represents a separate embodiment.

In various embodiments, the compound represented by the structure of formula I is a compound of formula Id:

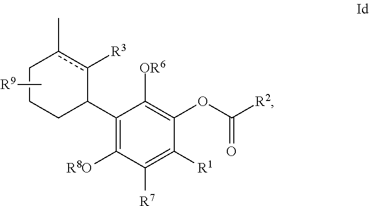

wherein
   R¹ is C₁-C₁₂ alkyl, C₂-C₁₂ alkenyl, or C₂-C₁₂ alkynyl;
   R² is C₁-C₁₀ alkyl, C₂-C₁₀ alkenyl, or C₂-C₁₀ alkynyl;
   R³ and R⁷ are each independently H, deuterium, hydroxyl, halogen, nitro, or cyano;
   R⁶ and R⁸ are each independently H or deuterium;
   R⁹ is C₁-C₄ alkyl or C₂-C₄ alkenyl; and
   the dotted line represents an optional second carbon-carbon bond, wherein each of the alkyl, alkenyl, or alkynyl is optionally deuterated,
including salts, hydrates, solvates, polymorphs, optical isomers, geometrical isomers, enantiomers, and diastereomers. Each possibility represents a separate embodiment.

Representative and non-limiting examples of such structures are compounds selected from the group consisting of compounds 1 to 33 with each possibility representing a separate embodiment.

### Chemical Definitions

An “alkyl” group refers to any unsubstituted or substituted alkyl. An “unsubstituted alkyl” group refers to a saturated aliphatic hydrocarbon, including straight-chain, branched-chain and cyclic alkyl groups. In one embodiment, the alkyl group has 1-12 carbons designated herein as C₁-C₁₂ alkyl. In another embodiment, the alkyl group has 1-10 carbons designated herein as C₁-C₁₀ alkyl. In another embodiment, the alkyl group has 1-6 carbons designated here in as C₁-C₆ alkyl. In another embodiment, the alkyl group has 1-4 carbons designated here in as C₁-C₄ alkyl. A “substituted alkyl” group refers to an alkyl which is substituted by one or more groups selected from halogen, hydroxyl, nitro, cyano, amino or amido. Each possibility represents a separate embodiment. Thus, it is to be understood that per-halogenated alkyls, such as trifluoromethyl, are considered to be alkyl groups in general, and substituted alkyl groups in particular (c.f., substituent R² of compound 8 of the present invention).

An “alkenyl” group refers to any unsubstituted or substituted alkenyl. An “unsubstituted alkenyl” group refers to an aliphatic hydrocarbon group containing at least one carbon-carbon double bond including straight-chain, branched-chain and cyclic alkenyl groups. In one embodiment, the alkenyl group has 2-12 carbon atoms designated here as C₂-C₁₂ alkenyl. In another embodiment, the alkenyl group has 2-6 carbon atoms in the chain designated here as C₂-C₆ alkenyl. Exemplary alkenyl groups include ethenyl, propenyl, n-butenyl, i-butenyl, 3-methylbut-2-enyl, n-pentenyl, heptenyl, octenyl, cyclohexyl-butenyl and decenyl. Each possibility represents a separate embodiment. The alkenyl group can be unsubstituted or substituted through available carbon atoms with one or more groups defined hereinabove for alkyl.

An “alkynyl” group refers to any unsubstituted or substituted alkynyl. An “unsubstituted alkynyl” group refers to an aliphatic hydrocarbon group containing at least one carbon-carbon triple bond including straight-chain and branched-chain. In one embodiment, the alkynyl group has 2-12 carbon atoms in the chain designated here as C₂-C₁₂ alkynyl. In another embodiment, the alkynyl group has 2-6 carbon atoms in the chain designated here as C₂-C₆ alkynyl. Exemplary alkynyl groups include ethynyl, propynyl, n-butynyl, 2-butynyl, 3-methylbutynyl, n-pentynyl, heptynyl, octynyl and decynyl. Each possibility represents a separate embodiment. The alkynyl group can be unsubstituted or substituted through available carbon atoms with one or more groups defined hereinabove for alkyl.

The term “aryl” used herein alone or as part of another group refers to an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. Each possibility represents a separate embodiment. The aryl group can be unsubstituted or substituted through available carbon atoms with one or more groups defined hereinabove for alkyl.

The term “heterocyclic ring” or “heterocycle” used herein alone or as part of another group refers to five-membered to eight-membered rings that have 1 to 4 heteroatoms, such as oxygen, sulfur and/or nitrogen, in particular oxygen, as a ring atom. These five-membered to eight-membered rings can be saturated, fully unsaturated or partially unsaturated, with fully saturated rings being preferred. The term “n-membered heterocycle” refers to a heterocycle, which has n ring atom that include both the ring carbon atoms and the ring heteroatoms. Exemplary heterocyclic rings include piperidinyl, pyrrolidinyl pyrrolinyl, pyrazolinyl, pyrazolidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, dihydrothiazolyl, glucosyl, and the like. Each possibility represents a separate embodiment. The heterocycle group can be unsubstituted or substituted through available atoms with one or more groups defined hereinabove for alkyl.

A “hydroxy” group refers to an —OH group.

A “halogen” refers to chlorine, bromine, fluorine, and/or iodine. Each possibility represents a separate embodiment.

A “haloalkyl” refers to an alkyl group having some or all of the hydrogens independently replaced by a halogen. Exemplary haloalkyls include, but are not limited to, trichloromethyl, tribromomethyl, trifluoromethyl, triiodomethyl, difluoromethyl, chlorodifluoromethyl, pentafluoroethyl, 1,1-difluoroethyl bromomethyl, chloromethyl, fluoromethyl, iodomethyl, and the like. Each possibility represents a separate embodiment.

A “nitro” group refers to an —NO₂ group.

A “cyano” group refers to a —CN group.

The term “deuterium” and “deuteron” as used herein are interchangeable and refer to the stable isotope of hydrogen, which contains a single proton and a single neutron in its nucleus. As used herein, the term “optionally deuterated” refers to a group that can be unsubstituted or substituted with at least one deuteron, i.e., one or more hydrogens can be replaced by one or more deuterons. In some embodiments, all hydrogens in the group are replaced by deuterons. Appearances of D, e.g., in the definition of X, and the chemical structures of compounds 9, 13, and 19 refer to the deuteron isotope.

All stereoisomers of the compounds of the present invention are contemplated, either in admixture or in pure or substantially pure form. These compounds can have asymmetric centers at one or more atoms. Consequently, the compounds can exist in enantiomeric or diastereomeric forms or in mixtures thereof. The present invention contemplates the use of any racemates (i.e., mixtures containing equal amounts of each enantiomer), enantiomerically enriched mixtures (i.e., mixtures enriched for one enantiomer), pure enantiomers or diastereomers, or any mixtures thereof. The chiral centers can be designated as R or S or R,S or d,D, I,L or d,l, D,L. Where compounds of the invention contain a double bond (for example where R¹ is a C₂-C₁₂ alkenyl), it is to be understood that it encompasses all structural and geometrical isomers including cis, trans, E and Z isomers and optical isomers, independently at each occurrence.

One or more of the compounds of the invention, may be present as a salt. The term “salt” as used herein encompasses both base and acid addition salts including, but not limited to, carboxylate salts or salts with amine nitrogens, and include salts formed with the organic and inorganic anions and cations detailed below. Further encompassed by the term are salts formed by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and the like. Each possibility represents a separate embodiment.

The term “organic or inorganic cation” refers to counter-ions for the carboxylate anion of a carboxylate salt. The counter-ions are chosen from the alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl) ammonium, phenylethylbenzylammonium, dibenzylethylenediammonium, and like cations. Each possibility represents a separate embodiment. Other cations encompassed by the above term include the protonated form of procaine, quinine and N-methylglucosamine. Each possibility represents a separate embodiment. Furthermore, any zwitterionic form of the instant compounds formed by a carboxylic acid and an amino group are also contemplated.

The present invention also includes solvates of any of compounds represented by formulae I, Ia, Ib, Ic, Id or any of compounds 1-33 and salts thereof. “Solvate” means a physical association of a compound of the invention with one or more solvent molecules. This physical association involves varying degrees of ionic and covalent bonding, including hydrogen bonding. In certain instances, the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of any of compounds represented by formulae I, Ia, Ib, Ic, Id or any of compounds 1-33 and salts thereof. The term “polymorph” refers to a particular crystalline state of a substance, which can be characterized by particular physical properties such as X-ray diffraction, IR spectra, melting point, and the like.

### Pharmaceutical Compositions

According to certain aspects and embodiments, the invention relates to a pharmaceutical composition comprising a therapeutically effective amount of a compound of formulae I, Ia, Ib, Ic, Id or 1-33 including salts, hydrates, solvates, polymorphs, optical isomers, geometrical isomers, enantiomers, and diastereomers, the pharmaceutical composition further comprising a pharmaceutically acceptable carrier or excipient.

Suitable pharmaceutically acceptable carriers or excipients include, but are not limited to, a binder, a filler, a diluent, a surfactant or emulsifier, a glidant or lubricant, buffering or pH adjusting agent, a tonicity enhancing agent, a wetting agent, a chelating agent, a preservative, an antioxidant, a flavoring agent, a colorant, and a mixture or combination thereof. Each possibility represents a separate embodiment.

Suitable binders include, but are not limited to, polyvinylpyrrolidone, copovidone, hydroxypropyl methylcellulose, starch, and gelatin. Each possibility represents a separate embodiment.

Suitable fillers include, but are not limited to, sugars such as lactose, sucrose, mannitol or sorbitol and derivatives therefore (e.g., amino sugars), ethylcellulose, microcrystalline cellulose, and silicified microcrystalline cellulose. Each possibility represents a separate embodiment.

Suitable lubricants include, but are not limited to, sodium stearyl fumarate, stearic acid, polyethylene glycol or stearates, such as magnesium stearate. Each possibility represents a separate embodiment.

Suitable diluents include, but are not limited to, dicalcium phosphate dihydrate, sugars, lactose, calcium phosphate, cellulose, kaolin, mannitol, sodium chloride, starch, and various oils. Each possibility represents a separate embodiment.

Suitable surfactants or emulsifiers include, but are not limited to, polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil, and lecithin. Each possibility represents a separate embodiment.

Suitable glidants or lubricants include, but are not limited to, colloidal silicon dioxide, magnesium stearate, talc, and mineral oil. Each possibility represents a separate embodiment.

Suitable buffering or pH adjusting agents include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide, and magnesium hydroxide. Each possibility represents a separate embodiment.

Suitable tonicity enhancing agents include, but are not limited to, ionic and non-ionic agents such as, alkali metal or alkaline earth metal halides, urea, glycerol, sorbitol, mannitol, propylene glycol, and dextrose. Each possibility represents a separate embodiment.

Suitable wetting agents include, but are not limited to, glycerin, cetyl alcohol, and glycerol monostearate. Each possibility represents a separate embodiment.

Suitable chelating agents include, but are not limited to, modified or unmodified cyclodextrin (e.g., α-cyclodextrin, β-cyclodextrin, γ-cyclodextrin, 2-hydroxypropyl-β-cyclodextrin, methyl-β-cyclodextrin), dextrin, maltodextrin, and a mixture or combination thereof. Each possibility represents a separate embodiment.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide. Each possibility represents a separate embodiment.

Suitable antioxidants include, but are not limited to, sorbic acid, ascorbic acid, ascorbate, glycine, α-tocopherol, butylated hydroxyanisole (BHA), and butylated hydroxytoluene (BHT). Each possibility represents a separate embodiment.

Suitable flavoring agents include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot. Each possibility represents a separate embodiment.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide. Each possibility represents a separate embodiment.

In certain aspects and embodiment, the pharmaceutical composition of the present invention is formulated as tablet, pill, capsule (e.g., soft or hard gelatin capsule), pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, ointment, suppository, a gel, and a cream. Each possibility represents a separate embodiment.

For preparing solid compositions such as tablets, the active pharmaceutical ingredient is mixed with a pharmaceutical carrier or excipient to form a solid pre-formulation composition containing a substantially homogeneous distribution of the compound of the present invention in the pharmaceutical carrier or excipient.

Any method can be used to prepare the pharmaceutical compositions. For example, solid dosage forms can be prepared by wet granulation, dry granulation, direct compression, and the like as is known in the art. The liquid forms in which the compounds of the present invention may be incorporated, for administration via a route selected from oral, topical or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Each possibility represents a separate embodiment.

The compositions of the present invention may be formulated as single-phase aqueous emulsion or multiple emulsions. According to some embodiments, the composition is formulated as an emulsion. These emulsions may be oil-in-water (o/w) emulsions, water-in-oil (w/o) emulsions, or multiple emulsions such as oil-in-water-in-oil (o/w/o) or water-in-oil-in-water (w/o/w) emulsions. It is understood that the oil phase can comprise silicone oils, non-silicone organic oils, or mixtures thereof. The compositions can comprise two immiscible phases that are reconstituted prior to use. Each possibility represents a separate embodiment of the present invention.

In certain embodiments, the compositions of the present invention are liposomal compositions comprising a compound represented by the structure of formula I or any of formulae Ia, Ib, Ic, Id and compounds 1-33 as defined herein encapsulated in a liposome comprising a lipid bilayer structure.

Another formulation employed in the methods of the present invention comprises transdermal delivery devices (“patches”). Such transdermal patches may be used to provide continuous or discontinuous infusion of the compound of the present invention in controlled amounts. The construction and use of transdermal patches for the delivery of pharmaceutical agents is well known in the art.

In yet another embodiment, the composition is prepared for topical administration, e.g., as an oil, ointment, gel or cream. Adjuvants for topical administration may include, for example, sodium carboxymethylcellulose, polyacrylates, polyoxyethylene-polyoxypropylene-block polymers, polyethylene glycol and wood wax alcohols. The term “gel” as used herein, refers to a substantially dilute cross-linked system, which exhibits little or no flow when in the steady-state having a solid jelly-like matrix. As contemplated herein, gel may comprise hydrogel, organogel, thermosensitive gel, non-thermosensitive gel, and aerogel. Each possibility represents a separate embodiment.

Compositions for inhalation or aspiration include solutions and suspensions in pharmaceutically acceptable aqueous or organic solvents, or mixtures thereof, as well as powders. The liquid or solid compositions may contain suitable pharmaceutically acceptable carriers or excipients as described above. The compositions may be administered by the oral or nasal respiratory route. Compositions may also be nebulized by use of inert gases. Nebulized solutions may be breathed directly from the nebulizing device or the nebulizing device may be attached to a face mask tent, or intermittent positive pressure breathing machine. Solution, suspension, or powder compositions may be administered, preferably orally or nasally, from devices (e.g., inhalers) that deliver the formulation in an appropriate manner.

The pharmaceutical compositions of the present invention may exhibit release mode which may be immediate release, controlled release or a mixture thereof. Each possibility represents a separate embodiment of the invention. “Immediate release” (IR) compositions in the context of the present invention refers to compositions in which the active ingredient is released without delay following administration. “Controlled release” (CR) compositions in the context of the present invention refers to compositions in which the active ingredient is released gradually over a period of time following administration.

### Therapeutic Uses

The pharmaceutical composition comprising a therapeutically effective amount of at least one compound according to the present invention and a pharmaceutically acceptable carrier or excipient can be used as a medicament. Thus, there is provided a pharmaceutical composition comprising a therapeutically effective amount a compound of formulae I, Ia, Ib, Ic, Id or 1-33 including salts, hydrates, solvates, polymorphs, optical isomers, geometrical isomers, enantiomers, and diastereomers; and further comprising a pharmaceutically acceptable carrier or excipient for use as a medicament.

In some aspects and embodiments, the compounds and pharmaceutical compositions comprising same are useful for treating an estrogen receptor (ER)-related disease or disorder.

The term “estrogen receptor (ER)-related disease or disorder” refers to any disease, condition, disorder, pathology, or a combination thereof, wherein an estrogen receptor (ER), such as ER a, ER B, or both, is involved in, induces, initiates, propagates, determines, or any combination or equivalent thereof, in the pathogenesis, pathophysiology, or both. Non-limiting examples of ER-related diseases or disorders include breast cancer, ovarian cancer, uterine serous carcinoma, colon cancer, prostate cancer, polycystic ovary syndrome, endometrial cancer, endometriosis, fibrosis, dysmenorrhea, precocious puberty, and gynecomastia. Each possibility represents a separate embodiment. The compounds and compositions of the present invention can be utilized for treating an inflammatory disease or disorder, in particular, inflammatory bowel disease (IBD).

The term “inflammatory disease or disorder” refers to a disease, condition or disorder associated with inflammation. The term “inflammation” as used herein refers the process by which a subject's immune system coordinates a response to tissue damage, infection, antigenic challenge, etc. Inflammation may be associated with an increased blood supply to the tissue, increased capillary permeability in the tissue and/or increased leukocyte migration to the tissue. The compounds and compositions of the present invention can be utilized in treating a disease or disorder associated with excess release of inflammatory mediators and chemokines including, but not limited to, monocyte chemotactic protein-1 (MCP-1).

The term “inflammatory bowel disease (IBD)” refers to chronic inflammation of the unknown origin occurring in the intestine. Non-limiting examples of IBD include Crohn's disease, granulomatous colitis, lymphocyte colitis, collagenous colitis, ulcrerative colitis, diversion colitis, and coeliac disease. Each possibility represents a separate embodiment.

In some aspects and embodiments, there is provided a pharmaceutical composition comprising a therapeutically effective amount of (E)-3-(3,7-dimethylocta-2,6-dien-1-yl)-6-hexyl-2,4-dihydroxyphenyl acetate for use in treating inflammatory bowel disease (IBD). In other aspects and embodiments, there is provided a method of treating inflammatory bowel disease (IBD), the method comprising administering to a subject in need thereof a pharmaceutical composition comprising a therapeutically effective amount of (E)-3-(3,7-dimethylocta-2,6-dien-1-yl)-6-hexyl-2,4-dihydroxyphenyl acetate. In further aspects and embodiments, there is provided the use of (E)-3-(3,7-dimethylocta-2,6-dien-1-yl)-6-hexyl-2,4-dihydroxyphenyl acetate for the preparation of a medicament for treating inflammatory bowel disease (IBD).

As used herein, the term “treating” includes, but is not limited to, preventing the disorder or disease from occurring in a subject or arresting the development or progression of the disorder or disease, relieving the disorder or disease, causing regression of the disorder or disease, relieving a condition caused by the disease or disorder, or ameliorating the symptoms of the disease or disorder. With reference to IBD, the term “treating” includes, but is not limited to, alleviating, suppressing, preventing, delaying the onset and/or attenuating the progression of the disease or at least one symptom of IBD after the onset of the disease. Common symptoms after the onset of IBD include, but are not limited to, diarrhea, abdominal pain and cramping, blood in the stool, ulcer colon shortening, and/or histomorphological changes, as well as reduced appetite and weight loss. According to some embodiments, “treating” encompasses reduction or even prevention of inflammatory relapses associated with IBD.

As used herein, the term “administering” refers to bringing in contact with the compound and/or composition of the present invention. Administration can be accomplished to living organisms, for example humans.

A “therapeutic” treatment is a treatment administered to a subject who exhibits signs of pathology for the purpose of diminishing or eliminating those signs. A “therapeutically effective amount” is that amount of compound which is sufficient to provide a beneficial effect to the subject to which the compound is administered. The precise dose to be employed in the pharmaceutical composition comprising a compound of any of formulae I, Ia, Ib, Ic, Id or 1-33 will depend on the route of administration, and the seriousness of the disease, and should be decided according to the judgment of the practitioner and each patient's circumstances. A preferred dosage will be within the range of about 0.01-1,000 mg/kg of body weight, about 0.1 mg/kg to 100 mg/kg, about 1 mg/kg to 100 mg/kg, about 10 mg/kg to 75 mg/kg, about 0.1 to 1 mg/kg etc., including each value within the specified ranges. Exemplary non-limiting amounts of the compound of any of formulae I, Ia, Ib, Ic, Id or 1-33 include about 0.1 mg/kg, about 0.2 mg/kg, about 0.5 mg/kg, about 1 mg/kg, about 5 mg/kg, about 10 mg/kg, about 20 mg/kg, about 50 mg/kg, about 60 mg/kg, about 75 mg/kg, and about 100 mg/kg. Each possibility represents a separate embodiment.

Alternatively, the amount administered can be measured and expressed as molarity of the administered compound. By way of illustration and not limitation, the compound of any of formulae I, Ia, Ib, Ic, Id or 1-33 can be administered in a range of about 0.001 to 10 mM, including each value within the specified range e.g., about 0.01, 0.1, 0.25, 0.5, 1 or 2 mM. Each possibility represents a separate embodiment. Alternatively, the amount administered can be measured and expressed as mg/ml, μg/ml, or ng/ml.

The administration schedule will depend on several factors such as the severity and progression of the disorder, age, weight etc. For example, the compositions of the invention can be taken once-daily, twice-daily, thrice daily, once-weekly or once-monthly. In addition, the administration can be continuous, i.e., every day, or intermittent. The terms “intermittent” or “intermittently” as used herein means stopping and starting at either regular or irregular intervals. For example, intermittent administration can be administration one to six days per week or it may mean administration in cycles (e.g., daily administration for two to eight consecutive weeks, then a rest period with no administration for up to one week) or it may mean administration on alternate days. The effectiveness of said compositions could enable a shortened period of treatment with superior results.

Although the pharmaceutical composition comprising a compound of any of formulae I, Ia, Ib, Ic, Id or 1-33 may be administered as the single therapeutic agent, combination therapy including co-administration with one or more additional agents is within the scope of the present invention. Co-administration of a compound of any of formulae I, Ia, Ib, Ic, Id or 1-33 with one or more therapeutic agents may take place sequentially in any order, simultaneously or a combination thereof. For example, administration of a compound of any of formulae I, Ia, Ib, Ic, Id or 1-33 can take place prior to, after or at the same time as the administration of the additional therapeutic agent(s). For example, a total treatment period can be decided for the compound of any of formulae I, Ia, Ib, Ic, Id or 1-33. The additional agent(s) can be administered prior to the onset of treatment with the compound of any of formulae I, Ia, Ib, Ic, Id or 1-33 or following treatment with the compound of any of formulae I, Ia, Ib, Ic, Id or 1-33. In addition, the additional agent(s) can be administered during the period of administering the compound of any of formulae Ia, Ib, Ic, Id or 1-33 but does not need to occur over the entire treatment period. In another embodiment, the treatment regimen includes pre-treatment with one agent, followed by the addition of the other agent or agents. Alternating sequences of administration are also contemplated. Alternating administration includes administration of a compound of any of formulae I, Ia, Ib, Ic, Id or 1-33, followed by the additional agent, followed by a compound of any of formulae I, Ia, Ib, Ic, Id or 1-33, etc. The aforementioned sequences can also be administrated in several cycles wherein each cycle may be similar or different with each possibility representing a separate embodiment. The therapeutic efficacy of the combination of the compound of any of formulae I, Ia, Ib, Ic, Id or 1-33 and the additional agent(s) is at least additive. In some embodiments, the therapeutic efficacy is synergistic, namely the overall dose of each of the components may be lower, thus resulting in significantly lower side effects experienced by the subject, while a sufficient desirable therapeutic effect is nonetheless achieved. When combination therapy is involved, the compound of any of formulae I, Ia, Ib, Ic, Id or 1-33 and the additional therapeutic agent(s) may be provided in a single dosage form such as a fixed-dose combination or in separate compositions intended for simultaneous administration.

The one or more additional therapeutic agents include, but are not limited to, cannabinoids, terpenes, terpenoids, flavonoids, oils, nitrogenous compounds, amino acids, proteins, glycoproteins, sugars, hydrocarbons, fatty acids, esters, lactones, steroids, non-cannabinoid phenols, and a mixture or combination thereof. Each possibility represents a separate embodiment.

In some aspects and embodiments, the compounds and compositions of the present invention are administered in combination with at least one cannabinoid. Exemplary cannabinoids that can be used in the combination therapy include, but are not limited to, cannabidivarinic acid (CBDVA), cannabidiolic acid (CBDA), cannabigerolic acid (CBGA), cannabidiol (CBD), cannabinol (CBN), cannabinolic acid (CBNA), tetrahydrocannabinol (THC), cannabichromene (CBC), cannabichromenic acid (CBCA), tetrahydrocannabinolic acid (THCA), cannabicitran, tetrahydrocannabivarin (THCV), cannabigerol (CBG), sesquicannabigerol (sesqui-CBG), sesquicannabigerolic acid (sesqui-CBGA), CBGA-C4, CBG-C4, cannabigerovarinic acid (CBGVA), cannabigerivarin (CBGV), cannabigerorcinic acid (CBGOA), cannabigerorcin (CBGO), cannabigerolic acid monomethyl ether (CBGMA), cannabigerol monomethyl ether (CBGM), cannabicyclol (CBL), cannabicyclolic acid (CBLA), THCA-C4, THC-C4, tetrahydrocannabivarin carboxylic acid (THCVA), tetrahydrocannabivarin (THCV), tetrahydrocannabiorcolic acid (THCOA), tetrahydrocannabiorcol (THCO), THCMA, THCM, CBDA-C4, CBD-C4, cannabidiorcolic acid (CBDOA), cannabidiorcol (CBDO), cannabidiolic acid monomethyl ether (CBDMA), cannabidiol monomethylether (CBDM), cannabichromenic acid (CBCA), cannabichromene (CBC), cannabichromevarinic acid (CBCVA), cannabichromevarin (CBCV), cannabiorchromenic acid (CBCOA), cannabiorchromene (CBCO), cannabinolic acid (CBNA), cannabinol (CBN), cannabinol-C4 (CBN-C4), cannabivarinic acid (CBNVA), cannabivarin (CBNV), cannabiorcolic acid (CBNOA), cannabiorcol (CBNO), CBNA-8-OH, CBN-8-OH, cannabinol methylether (CBNM), cannabielsoin acid (CBEA), cannabielsoin (CBE), cannabielsoic acid (CBEVA), cannabielsoin (CBEV), cannabinodiolic acid (CBNDA), cannabinodiol (CBND), cannabinodivarinic acid (CBNDVA), (−)-Δ⁸-trans-tetrahydrocannabinol (Δ⁸-THC), cannabitriol-1 (CBT-1), CBT-2, CBT-3, CBTA-1, CBTA-3, cannabitriolvarin (CBTV), CBTV-3, epicannabidiol, and a mixture or combination thereof. Each possibility represents a separate embodiment. Typical ratios of the compound of the present invention and the at least one cannabinoid include, but are not limited to, about 1:1,000 to about 1,000:1, including all iterations of ratios within the specified range. Exemplary ratios include, but are not limited to, about 1:1,000, about 1:900, about 1:800, about 1:700, about 1:600, about 1:500, about 1:400, about 1:300, about 1:200, about 1:100, about 1:75: about 1:50, about 1:40, about 1:30, about 1:20, about 1:15, about 1:10, about 1:5, about 1:2, about 1:1, about 2:1, about 5:1, about 10:1, about 15:1, about 20:1, about 30:1, about 40:1, about 50:1, about 75:1, about 100:1, about 200:1, about 300:1, about 400:1, about 500:1, about 600:1, about 700:1, about 800:1, about 900:1, or about 1,000:1. Each possibility represents a separate embodiment.

In other aspects and embodiments, the compounds and compositions of the present invention are administered in combination with an ER activation inhibitor. ER activation inhibitors within the scope of the present invention include, but are not limited to, aromatase inhibitor (AI), selective ER degrader (SERD), and a selective ER modulator (SERM) such as e.g., tamoxifen, toremifene, and ospemifene. Each possibility represents a separate embodiment.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the use of “a” and “an” means “at least one” or “one or more” unless the context clearly dictates otherwise.

As used herein, when a numerical value is preceded by the term “about”, the term “about” is intended to indicate ±10%.

As used herein, the term “and” or the term “or” are generally employed in its sense including “and/or” unless the context clearly dictates otherwise.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1: Synthesis

### Preparation of (E)-3-(3,7-dimethylocta-2,6-dien-1-yl)-6-pentyl-2,4-dihydroxyphenyl acetate

(E)-3-(3,7-dimethylocta-2,6-dien-1-yl)-6-hexyl-2,4-dihydroxyphenyl acetate was prepared in 5 steps:

### Step 1:

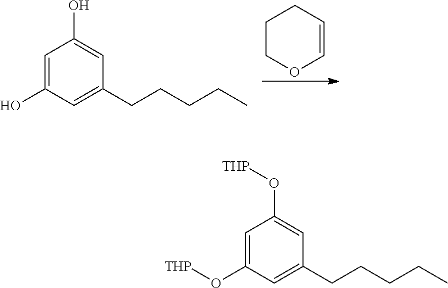

1-2 drops of H₃PO₄ (85%) were added to a solution of olivetol (3.0 g, 0.0167 mol) in DHP (3.56 g, 0.042 mol, 2.54 eq). The reaction mixture was stirred overnight at RT. The reaction mixture was then diluted with TBME (50 ml) and the organic solution was washed with 2N NaOH solution (50 ml), brine (50 ml×2) and evaporated to dryness. The product was purified by flash column chromatography, eluent-DCM/hexane to give 4.3 g of the pure product.

### Step 2:

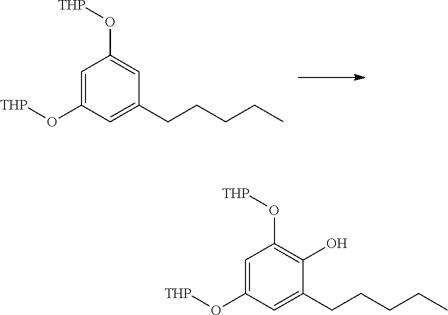

The pure product of step 1 (6.0 g, 0.0172 mol) was dissolved in DCM (170 ml) and a solution of NaHCO₃ (6.3 g, 0.0516 mol, 3 eq) in water (130 ml) was added. The emulsion was cooled to 0-5° C. and 3-chloro-perbenzoic acid (77% assay, 5.8 g, 0.025 mol, 1.5 eq) was added. The reaction mixture was stirred for 6.5-7 h at 0-5° C. and the organic layer was then separated. The organic layer was washed with brine and dried over sodium sulfate. The solvent was evaporated to dryness and the crude product was purified by flash column chromatography, eluent-DCM/hexane to give 1.2 g of the pure product and 2.1 g of the starting material (Di-THP-olivetol).

### Step 3:

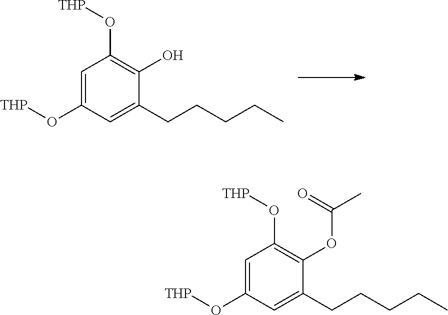

The pure product of step 2 (1.2 g, 3.3 mmol) was dissolved in pyridine (12 ml) and acetic anhydride (2.5 ml) was added. The reaction mixture was stirred for 2 h at RT and then was diluted with hexane (50 ml). The organic solution was then washed with 1N HCl (100 ml), water, brine and dried over sodium sulfate. The organic solution was subsequently evaporated to dryness to give 1.25 g of the crude product, which was purified by flash column chromatography, eluent-DCM/hexane to give 1.1 g of the pure product.

### Step 4:

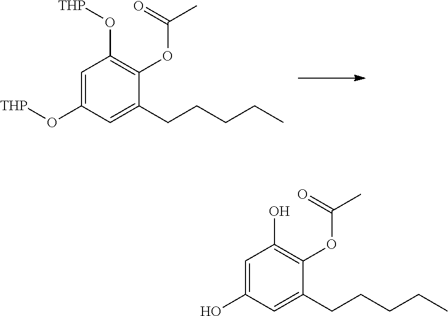

The pure product of step 3 (1.1 g, 2.7 mmol) was dissolved in methanol (12 ml) and p-TSA (20-25 mg) was added. The reaction mixture was stirred for 1 h. The reaction mixture was then diluted with water (50 ml) and the product was extracted with TBME (30 ml×3). The organic layers were combined and washed with brine. After evaporation, 0.8 g of the product were obtained and subsequently used without purification.

### Step 5:

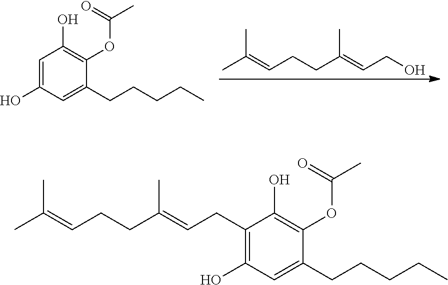

The product of step 4 (0.8 g, 3.4 mmol) and geraniol (0.52 g, 3.4 mmol) were dissolved in DCM (28 ml) and p-TSA (32 mg, 5% eq) was added. The reaction mixture was stirred for 7 h at RT under nitrogen, diluted with DCM (30 ml) and washed with water (30 ml). The solution was dried over sodium sulfate and DCM was evaporated to dryness to give 1.8 g as yellow oil. The oil was dissolved in hexane (5 ml) and the solution was stirred for 15-20 min at RT. A white solid was formed. The suspension was stirred for 1 h at 0-5° C. and a white solid was filtered, washed with cold hexane (3 ml) and dried. HPLC analysis: C.P.=88.5%.

¹H NMR (400 MHZ, CDCl₃) δ 6.26 (1H, Ar), 5.32 (1H, —OH), 5.26 (1H, C═CH), 5.15 (1H, —OH), 5.04 (1H, C═CH), 3.41 (2H), 2.37 (2H), 2.32 (3H), 2.06-2.09 (4H), 1.79 (3H), 1.67 (3H), 1.59 (3H), 1.52 (2H), 1.3 (4H), 0.88 (3H).

### Preparation of a Compound of Formula 6:

A compound of formula 6 was prepared in 5 steps:

### Step 1:

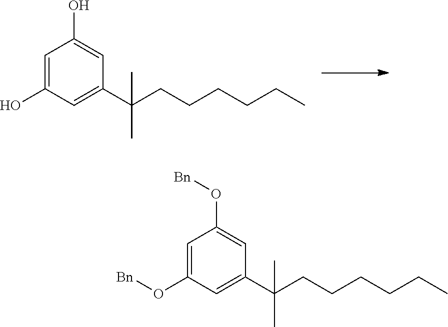 [SMILES: CCCCCCC(C)(C)c1cc(O)cc(O)c1 ; CCCCCCC(C)(C)c1cc(OCc2ccccc2)cc(OCc2ccccc2)c1]

Dimethyl-heptyl-olivetol (1.2 g, 5.1 mmol) and potassium carbonate (1.8 g, 12.7 mmol, 2.5 eq) were stirred in DMF (6 ml) for 30 min under nitrogen followed by the addition of benzyl bromide (2.2 g, 12.7 mmol, 2.5 eq). The reaction mixture was stirred overnight at RT and diluted with 1N HCl (60 ml). The product was extracted with hexane (50 ml, 20 ml). The organic solution was washed with water (50 ml×2) and dried over sodium sulfate. The crude product was purified by flash column chromatography, eluent-DCM/hexane to give 2.0 g of the pure product.

### Step 2:

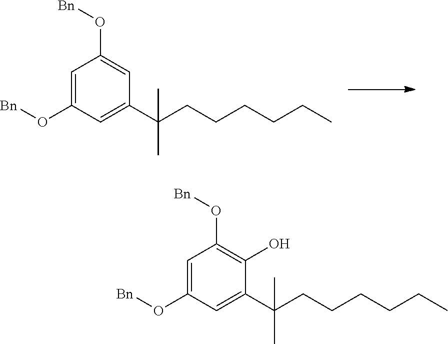 [SMILES: CCCCCCC(C)(C)c1cc(OCc2ccccc2)cc(OCc2ccccc2)c1 ; CCCCCCC(C)(C)c1cc(OCc2ccccc2)cc(OCc2ccccc2)c1O]

The pure product of step 1 (2.0 g) was oxidated with 3-chloro-perbenzoic acid as described in Step 2 of the preparation of (E)-3-(3,7-dimethylocta-2,6-dien-1-yl)-6-hexyl-2,4-dihydroxyphenyl acetate to give 0.3 g of the product and 1.0 g of the starting material.

### Step 3:

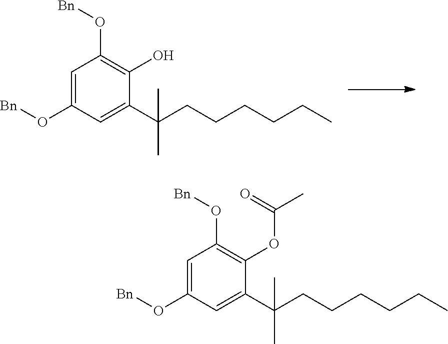 [SMILES: CCCCCCC(C)(C)c1cc(OCc2ccccc2)cc(OCc2ccccc2)c1O ; CCCCCCC(C)(C)c1cc(OCc2ccccc2)cc(OCc2ccccc2)c1OC(C)=O]

The product of step 2 (0.3 g) was acylated with acetic anhydride as described in Step 3 of the preparation of (E)-3-(3,7-dimethylocta-2,6-dien-1-yl)-6-hexyl-2,4-dihydroxyphenyl acetate to give 0.4 g of the pure product.

### Step 4:

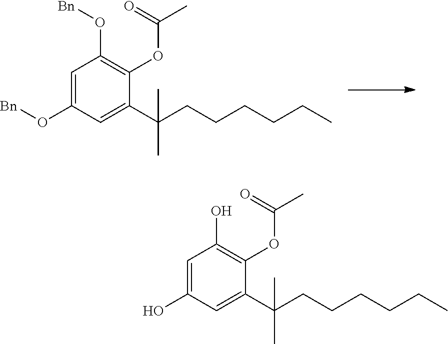 [SMILES: CCCCCCC(C)(C)c1cc(OCc2ccccc2)cc(OCc2ccccc2)c1OC(C)=O ; CCCCCCC(C)(C)c1cc(O)cc(O)c1OC(C)=O]

The product of step 3 (0.4 g) was dissolved in ethanol (CP, 5 ml) and nitrogen was passed through for 5-10 min. Pd/C (10%, 40 mg) was added to the reaction mixture and hydrogen was bubbled for 2 h at RT. The reaction mixture was filtered from the celite and ethanol was evaporated to dryness to give 0.27 g of the pure product.

### Step 5:

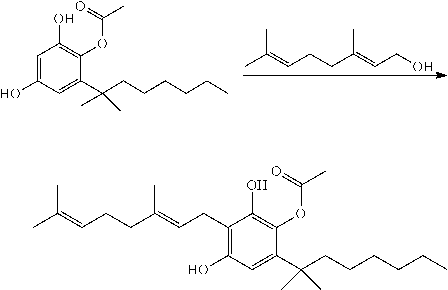

The product of step 4 (0.27 g) and geraniol (0.14 g) were coupled in DCM as described in Step 5 of the preparation of (E)-3-(3,7-dimethylocta-2,6-dien-1-yl)-6-hexyl-2,4-dihydroxyphenyl acetate. The crude product was purified by flash column chromatography, eluent-DCM/hexane to give 185 mg of the pure compound of formula 6.

### Preparation of a Compound of Formula 17:

A compound of formula 17 was prepared by the following scheme:

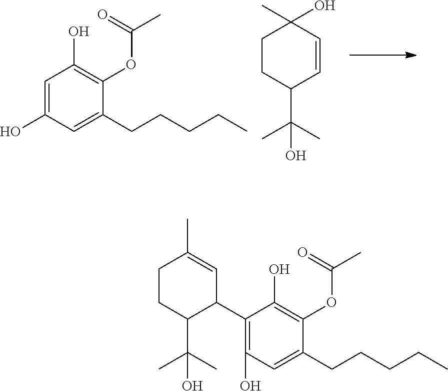

In particular, 1,8-diol (0.34 g, 2.0 mmol, prepared as described in WO 2005/100333) and the product of Step 4 of the preparation of (E)-3-(3,7-dimethylocta-2,6-dien-1-yl)-6-hexyl-2,4-dihydroxyphenyl acetate (0.48, 2.0 mmol) in DCM (20 ml) were mixed with p-TSA (20 mg, 5% eq) and stirred overnight at RT under nitrogen. The reaction mixture was diluted with DCM (20 ml) and washed with water (30 ml). The solution was dried over sodium sulfate and DCM was evaporated to dryness to give 0.75 g of the crude product as yellow oil. The crude product was purified by flash column chromatography, eluent-DCM/hexane to give 0.3 g of pure compound 17.

¹H NMR (400 MHZ, CDCl₃) δ 6.25 (1H, Ar), 5.44 (1H), 4.0 (3H), 2.81 (1H), 2.72 (1H), 2.4 (1H), 2.12 (2H), 1.97 (1H0, 1.77 (3H), 1.6 (1H), 1.57 (1H), 1.51 (2H), 1.3 (4H), 1.16-1.18 (6H), 0.88 (3H).

### Preparation of a Compound of Formula 15:

A compound of formula 15 was prepared according to the following scheme:

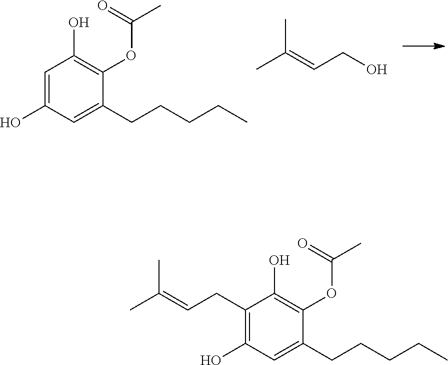

In particular, 3-methyl-2-buten-1-ol (0.172 g, 2.0 mmol), the product of Step 4 of the preparation of (E)-3-(3,7-dimethylocta-2,6-dien-1-yl)-6-hexyl-2,4-dihydroxyphenyl acetate (0.48 g, 2.0 mmol) and p-TSA (20 mg) in DCM (14 ml) were stirred overnight at RT under nitrogen. The reaction mixture was diluted with DCM (30 ml) and washed with water (30 ml). The solution was dried over sodium sulfate and DCM was evaporated to dryness to give 1.8 g as yellow oil. The oil was dissolved in hexane (14 ml) at reflux and the solution was cooled to RT. A white solid was formed. The suspension was stirred for 1 h at 0-5° C. and the white solid was filtered, washed with cold hexane (3 ml) and dried. The crude product was purified by flash column chromatography, eluent-DCM/hexane to give 90 mg pure compound 15.

### Example 2: Acute Inflammation

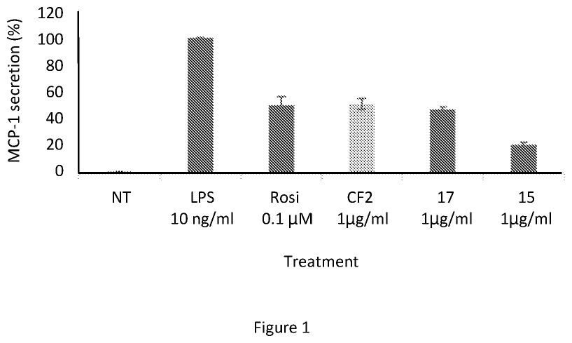

The efficacy of the compounds according to certain embodiments of the present invention in the treatment of IBD was assessed. Human colon epithelial cells were thawed and cultured in suitable media at a final concentration of 4×10⁵ cells per mL. The cells were then cultured and tested in triplicates for 48 hours. Following incubation, controls and test compounds were diluted in culture media to achieve the final assay concentration. Cells were treated with 10 ng/ml LPS with or without positive control/test compounds and incubated for 24 hours. Rosiglitazone at 7.2 μg/ml served as a positive control. Following incubation, the supernatants were collected, and the secretion levels of MCP-1 were measured by ELISA (R&D systems). Optical density was determined by ‘CLARIOStar-plus’® plate-reader. The chemokine concentration (pg/ml) was calculated using a standard curve. The results are shown in FIG. 1.

The results show that (E)-3-(3,7-dimethylocta-2,6-dien-1-yl)-6-hexyl-2,4-dihydroxyphenyl acetate, a compound of formula 15 and a compound of formula 17 effectively inhibited MCP-1 secretion in a statistically significant manner with compound 15 demonstrating the most pronounced inhibitory effect on MCP-1 secretion. Since MCP-1 plays a pivotal role in the manifestation of intestinal inflammation, the inhibition of MCP-1 secretion is indicative of the therapeutic efficacy of the compound of the invention in the treatment of IBD, in particular, colitis.

### Example 3: ER-Related Disease or Disorder

The efficacy of the compounds according to certain embodiments of the present invention in the treatment of ER-related diseases and disorder is assessed. Human adrenal cells are thawed and cultured in suitable media at a final concentration of 5×10⁵ cells per mL and tested in triplicates. Following 24 h incubation, cells are treated with 10 μM Forskolin. Following suitable incubation, cells are treated with positive control or test compounds. Next, supernatants are collected, and the secretion levels of sex hormones (testosterone & estradiol) are measured by ELISA. Optical density is determined by ‘CLARIOStar-plus’® plate-reader. Data are analyzed according to manufacturer instructions.

While certain embodiments of the invention have been illustrated and described, it is to be clear that the invention is not limited to the embodiments described herein. Numerous modifications, changes, variations, substitutions and equivalents will be apparent to those skilled in the art without departing from the spirit and scope of the present invention as described by the claims, which follow.

1-36. (canceled)

37. A compound represented by the structure of formula I: 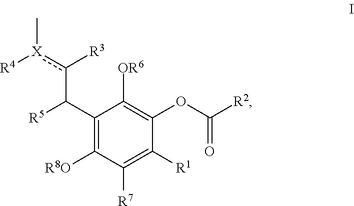
wherein
X is C, CH, or CD;
R¹ is C₁-C₁₂ alkyl, C₂-C₁₂ alkenyl, or C₂-C₁₂ alkynyl;
R² is C₁-C₁₀ alkyl, C₂-C₁₀ alkenyl, or C₂-C₁₀ alkynyl;
R³ and R⁷ are each independently H, deuterium, hydroxyl, halogen, nitro, or cyano;
R⁴ is H, deuterium, C₁-C₁₂ alkyl, C₂-C₁₂ alkenyl, or C₂-C₁₂ alkynyl;
R⁵ is H, deuterium, or R⁴ and R⁵ together form an aromatic or non-aromatic ring which is optionally substituted by C₁-C₄ alkyl, C₂-C₄ alkenyl, or hydroxyl;
R⁶ is H, deuterium, or R⁶ is absent and the oxygen attached thereto together with X form a six-membered heterocycle;
R⁸ is H, deuterium, or R⁸ together with R⁵ form a six-membered heterocycle which is optionally substituted by C₁-C₄ alkyl or C₂-C₄ alkenyl; and
the dotted line represents an optional second carbon-carbon bond, wherein each of the alkyl, alkenyl, or alkynyl is optionally deuterated, with the proviso that when X is C; R² is methyl; R³, R⁵, R⁶, R⁷, and R⁸ are each H; R⁴ is —CH₂CH₂CH═C(CH₃)₂; and the dotted line represents a second carbon-carbon bond, then R¹ is not n-pentyl,
including salts, hydrates, solvates, polymorphs, optical isomers, geometrical isomers, enantiomers, and diastereomers.

38. The compound of claim 37, wherein R¹ is C₁-C₁₂ alkyl; or wherein R¹ is selected from the group consisting of: propyl, butyl, pentyl, hexyl, heptyl, octyl, 1,1-dimethyl pentyl, 1-methyl pentyl, 1-methyl heptyl, 1,1-dimethyl heptyl, and 1-phenyl ethyl.

39. The compound of claim 37, wherein R² is methyl, ethyl or isopropyl; or wherein R² is trifluoromethyl; or wherein R² is CD₃; or wherein R² is 1-propylbutyl.

40. The compound of claim 37, wherein R³ is hydrogen or deuterium.

41. The compound of claim 37, wherein R⁴ is C₁-C₄ alkyl; or wherein R⁴ and R⁵ together form a terpineol or a limonene.

42. The compound of claim 37, wherein R⁶ is hydrogen; or wherein R⁶ is absent and the oxygen attached thereto together with X form a dihydropyran.

43. The compound of claim 37, wherein R⁷ is deuterium; or wherein R⁷ is halogen.

44. The compound of claim 37, wherein R⁸ is hydrogen; or wherein R⁸ together with R⁵ form a dihydropyran or a 2H-pyran.

45. The compound of claim 37, represented by the structure of formula Ia: 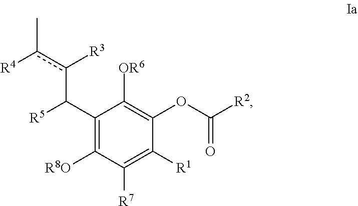
wherein
R¹ is C₁-C₁₂ alkyl, C₂-C₁₂ alkenyl, or C₂-C₁₂ alkynyl;
R² is C₁-C₁₀ alkyl, C₂-C₁₀ alkenyl, or C₂-C₁₀ alkynyl;
R³ and R⁷ are each independently H, deuterium, hydroxyl, halogen, nitro, or cyano;
R⁴ is H, deuterium, C₁-C₁₂ alkyl, C₂-C₁₂ alkenyl, or C₂-C₁₂ alkynyl;
R⁵, R⁶, and R⁸ are each independently H or deuterium; and
the dotted line represents an optional second carbon-carbon bond, wherein each of the alkyl, alkenyl, or alkynyl is optionally deuterated.

46. The compound of claim 37, represented by the structure of formula Ib: 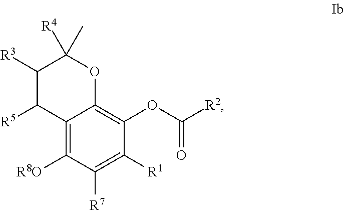
wherein
R¹ is C₁-C₁₂ alkyl, C₂-C₁₂ alkenyl, or C₂-C₁₂ alkynyl;
R² is C₁-C₁₀ alkyl, C₂-C₁₀ alkenyl, or C₂-C₁₀ alkynyl;
R³ and R⁷ are each independently H, deuterium, hydroxyl, halogen, nitro, or cyano;
R⁴ is H, deuterium, C₁-C₁₂ alkyl, C₂-C₁₂ alkenyl, or C₂-C₁₂ alkynyl; and
R⁵ and R⁸ are each independently H or deuterium;
wherein each of the alkyl, alkenyl, or alkynyl is optionally deuterated.

47. The compound of claim 37, represented by the structure of formula Ic: 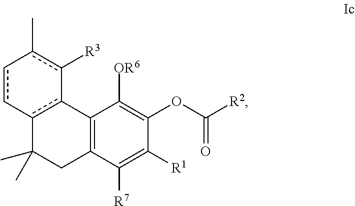
wherein
R¹ is C₁-C₁₂ alkyl, C₂-C₁₂ alkenyl, or C₂-C₁₂ alkynyl;
R² is C₁-C₁₀ alkyl, C₂-C₁₀ alkenyl, or C₂-C₁₀ alkynyl;
R³ and R⁷ are each independently H, deuterium, hydroxyl, halogen, nitro, or cyano;
R⁶ is H or deuterium; and
the dotted lines represent optional second carbon-carbon bonds, wherein each of the alkyl, alkenyl, or alkynyl is optionally deuterated.

48. The compound of claim 37, represented by the structure of formula Id: 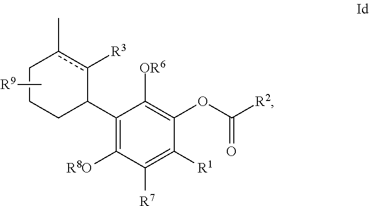
wherein
R¹ is C₁-C₁₂ alkyl, C₂-C₁₂ alkenyl, or C₂-C₁₂ alkynyl;
R² is C₁-C₁₀ alkyl, C₂-C₁₀ alkenyl, or C₂-C₁₀ alkynyl;
R³ and R⁷ are each independently H, deuterium, hydroxyl, halogen, nitro, or cyano;
R⁶ and R⁸ are each independently H or deuterium;
R⁹ is C₁-C₄ alkyl or C₂-C₄ alkenyl; and
the dotted line represents an optional second carbon-carbon bond, wherein each of the alkyl, alkenyl, or alkynyl is optionally deuterated.

49. The compound of claim 37, selected from the group consisting of: 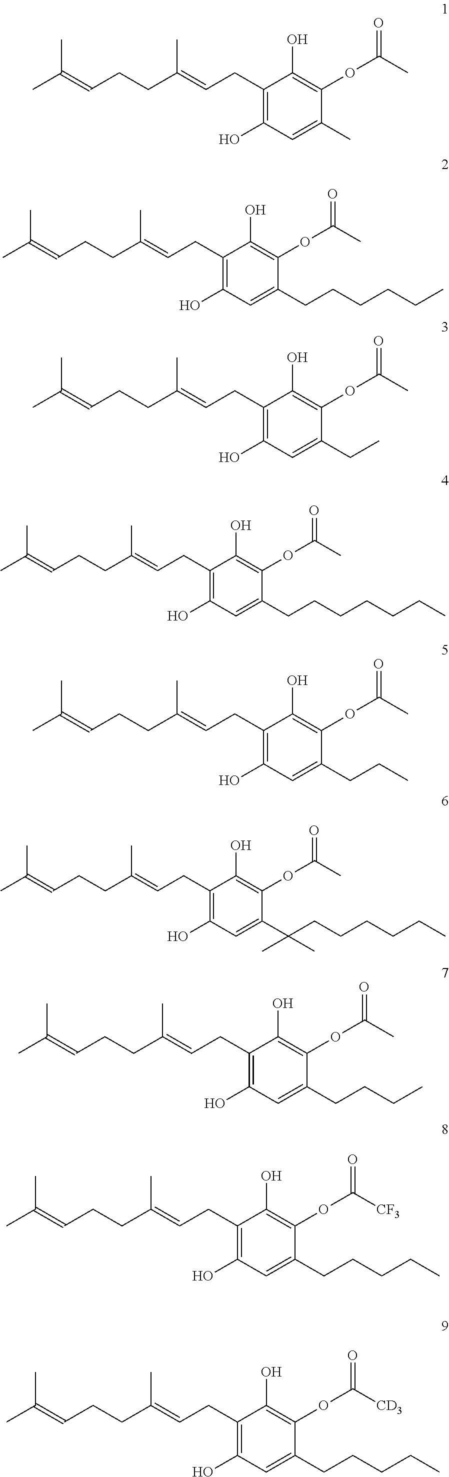 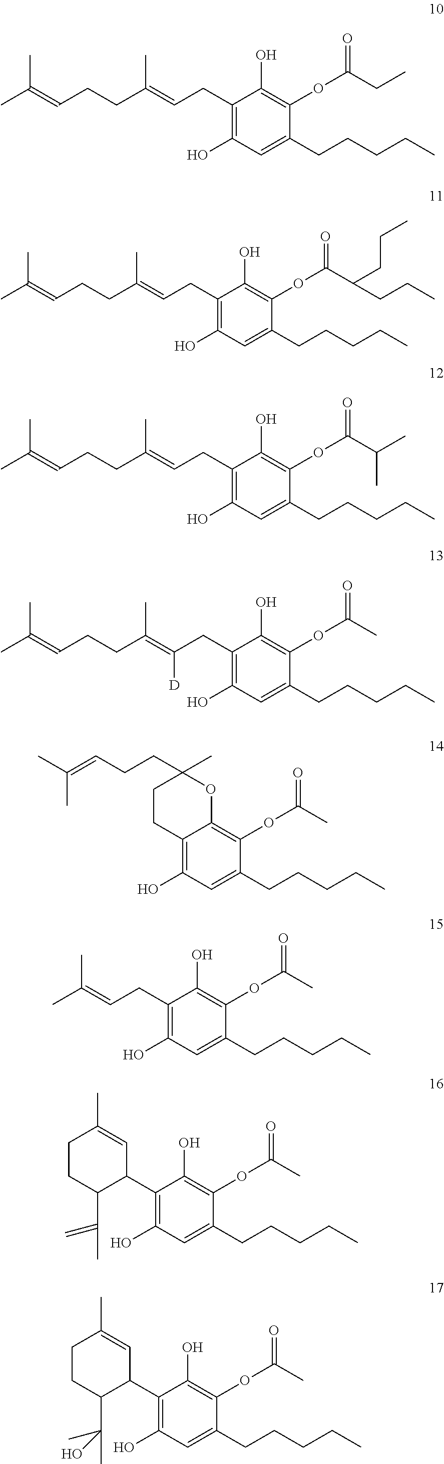 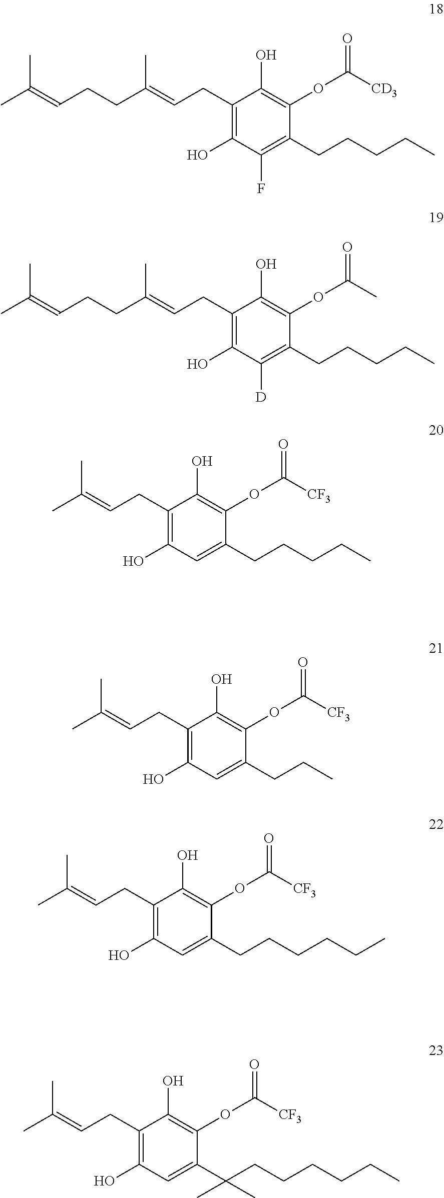 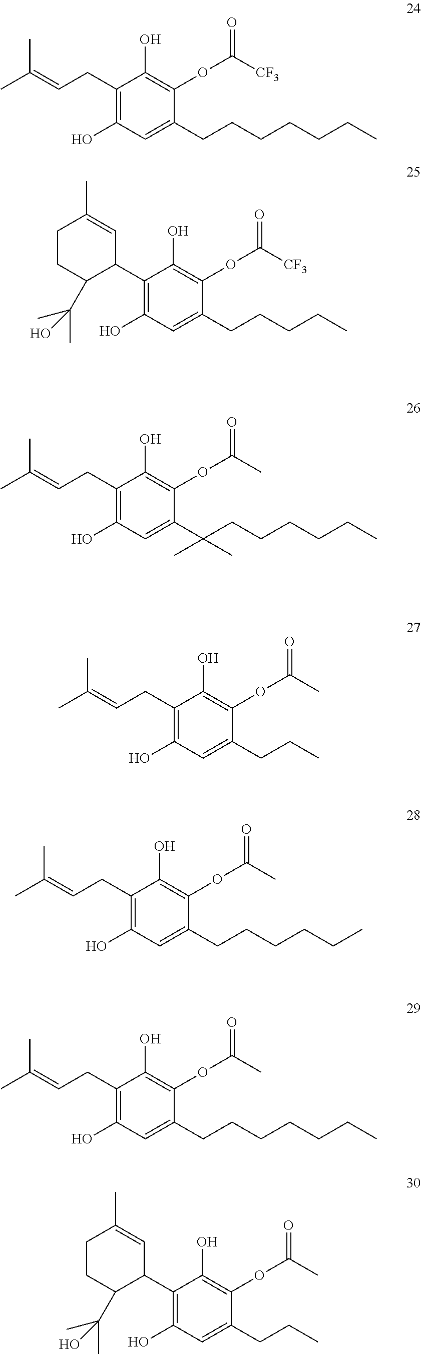 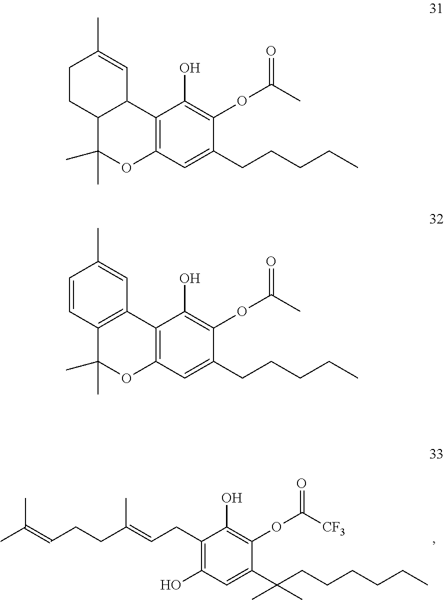
including salts, hydrates, solvates, polymorphs, optical isomers, geometrical isomers, enantiomers, and diastereomers.

50. A pharmaceutical composition comprising a therapeutically effective amount of at least one compound according to claim 37 and a pharmaceutically acceptable carrier or excipient.

51. The pharmaceutical composition of claim 50, wherein the pharmaceutically acceptable carrier or excipient comprises at least one of a binder, a filler, a diluent, a surfactant or emulsifier, a glidant or lubricant, a buffering or pH adjusting agent, a tonicity enhancing agent, a wetting agent, a chelating agent, a preservative, an antioxidant, a flavoring agent, a colorant, and a mixture or combination thereof.

52. The pharmaceutical composition of claim 50 in a form selected from the group consisting of: a tablet, pill, capsule, pellets, granules, powder, wafer, coated or uncoated beads, lozenge, sachet, cachet, elixir, osmotic pump, depot system, iontophoretic system, patch, suspension, dispersion, emulsion, solution, syrup, aerosol, oil, ointment, suppository, gel, and cream; or formulated for administration via a route selected from the group consisting of: oral, topical, transdermal, intra-arterial, sub-lingual, intranasal, intraperitoneal, intramuscular, subcutaneous, intravenous, and intra-alveolar.

53. A method of treating an estrogen receptor (ER)-related disease or disorder, the method comprising administering to a subject in need thereof a pharmaceutical composition comprising a therapeutically effective amount of at least one compound according to claim 37 and a pharmaceutically acceptable carrier or excipient.

54. The method of claim 53, wherein the disease or disorder is selected from the group consisting of: breast cancer, ovarian cancer, uterine serous carcinoma, colon cancer, prostate cancer, polycystic ovary syndrome, endometrial cancer, endometriosis, fibrosis, dysmenorrhea, precocious puberty, and gynecomastia.

55. A method of treating inflammatory bowel disease, the method comprising administering to a subject in need thereof a pharmaceutical composition comprising a therapeutically effective amount of at least one compound according to claim 37 and a pharmaceutically acceptable carrier or excipient.

56. The method of claim 55, wherein the inflammatory bowel disease is selected from the group consisting of: Crohn's disease, ulcerative colitis, granulomatous colitis, lymphocyte colitis, collagenous colitis, diversion colitis, and coeliac disease.
